(19) United States
(12) Patent Application Publication
(10) Pub. No.: US 2019/0117782 A1
(43) Pub. Date: Apr. 25, 2019

(54) CELLULARIZED HYDROGELS AND METHODS OF USING THE SAME

(71) Applicant: The Regents of the University of California, Oakland, CA (US)

(72) Inventors: David V. Schaffer, Danville, CA (US); Sanjay Kumar, Moraga, CA (US); Badriprasad Ananthanarayanan, Albany, CA (US); Tandis Vazin, Oakland, CA (US); Maroof M. Adil, Berkeley, CA (US)

(21) Appl. No.: 16/090,114

(22) PCT Filed: Apr. 4, 2017
(86) PCT No.: PCT/US17/25974

§ 371 Date: Sep. 28, 2018

Related U.S. Application Data
(60) Provisional application No. 62/318,516, filed on Apr. 5, 2016.

(51) Int. Cl. A61K 47/42; A61K 9/00; A61K 35/30; A61K 38/18; A61K 38/17; C12N 5/0793; A61K 9/06; A61K 47/10; A61K 47/36

(52) U.S. Cl. CPC A61K 47/42; A61K 9/0085; A61K 35/30; A61K 38/1833; A61K 38/185; A61K 38/177; C12N 2513/00; A61K 9/06; A61K 47/10; A61K 47/36; C12N 2533/30; C12N 2533/80; C12N 2533/50; C12N 5/0619

(57) ABSTRACT

The instant disclosure provides cellularized hydrogels containing cells encapsulated in linked polymers of hyaluronic acid, heparin and other components as described herein. Such cellularized hydrogels find use in variety of purposes including effective transplantation of cells into a host organism for cell therapy and the derivation of desired cell types. Other purposes include but are not limited to use as a tissue model for the in vitro study of cellular responses and behaviors. The instant disclosure also provides methods, including methods of making and using the described cellularized hydrogels. Also provided are kits that include components for making and/or using cellularized hydrogels e.g., according to the methods as described herein.

### CROSS-REFERENCE

This application claims the benefit of U.S. Provisional Patent Application No. 62/318,516, filed Apr. 5, 2016, which application is incorporated herein by reference in its entirety.

### STATEMENT REGARDING FEDERALLY SPONSORED RESEARCH

This invention was made with government support under Grant No. R01-N5074831 and Grant No. DP2OD004213 awarded by the National Institutes of Health. The government has certain rights in the invention.

### INTRODUCTION

Cell therapy, generally the administration of live cells or the maturation of specific cell populations in a subject for the treatment of a disease, has shown significant promise both in laboratory and clinical settings. For example, bone marrow transplantation, effectively a hematopoietic stem cell (HSC) transplant, has become a well-established protocol for the treatment of many diseases and disorders. However, the development of cell therapy for non-hematological diseases and deficiencies using cell types other than HSCs has encountered various hurdles including technical difficulties in cell delivery.

The effective delivery of significant numbers of viable cells that maintain or differentiate to a desired cell type and engraft at the target site is a primary component of any successful cell therapy. Establishing long-term survival and control of cell fate of transplanted cells as well as increasing the engraftment percentage of those cells delivered to a subject would greatly improve current cell therapies, including those directed to replacing neuronal cell types lost to neurodegenerative diseases. In some cell therapy paradigms, clinical effectiveness is hampered by ineffective dispersion of transplanted cells into the target tissues of the host. For example, in Parkinson's Disease poor cell therapy treatment outcomes have been attributed to low levels of integration of transplanted cells. In addition, localized engraftment, i.e., “hotspots”, resulting from ineffective dispersion of transplanted cells, has even been deemed responsible for undesirable side effects observed in certain cell therapy trials.

Furthermore, advanced cellularized hydrogels can aid in the generation of in vitro models of human diseases, including e.g., neurodegenerative and neurological diseases, by recapitulating micro-environmental influences on relevant cell types. For example, cell fate decisions, including those of adult neural stem and progenitor cells, can be better studied in appropriate three-dimensional cultures that allow the creation of neural networks and the identification of neural circuitry abnormalities, e.g., as displayed by diseased cells of relevant neurological disorders.

### SUMMARY

The instant disclosure provides cellularized hydrogels containing cells encapsulated in linked polymers of hyaluronic acid, heparin and other components as described herein. Such cellularized hydrogels find use in variety of purposes including effective transplantation of cells into a host organism for cell therapy and the derivation of desired cell types. Other purposes include but are not limited to use as a tissue model for the in vitro study of cellular responses and behaviors. The instant disclosure also provides methods, including methods of making and using the described cellularized hydrogels. Also provided are kits that include components for making and/or using cellularized hydrogels e.g., according to the methods as described herein.

Aspects of the instant disclosure include a method of delivering a cellularized hydrogel to a subject in need thereof by contacting a cellular sample with cell-encapsulating hydrogel components to generate a cellularized hydrogel mixture, wherein the cell-encapsulating hydrogel components comprise: (a) a first backbone polymer comprising hyaluronic acid with an attached azide or cyclooctyne reactive group; (b) a second backbone polymer comprising heparin with an attached azide or cyclooctyne reactive group; (c) a cell attachment peptide comprising an azide or cyclooctyne reactive group; and (d) a linking polymer comprising at least two azide reactive groups or at least two cyclooctyne reactive groups; incubating the cellularized hydrogel mixture under conditions sufficient to allow cross-linking of the first backbone polymer, the second backbone polymer, the cell attachment peptide and the linking polymer to produce a cellularized hydrogel; and injecting the cellularized hydrogel into an affected area of the subject, wherein the injecting results in the delivery of a therapeutically effective amount of the cells of the cellular sample into the treatment site of the subject.

In certain embodiments, the method includes, wherein the cell-encapsulating hydrogel components comprise: (a) a first backbone polymer comprising hyaluronic acid with an attached cyclooctyne reactive group; (b) a second backbone polymer comprising heparin with an attached cyclooctyne reactive group; (c) a cell attachment peptide comprising an azide reactive group; and (d) a linking polymer comprising at least two azide reactive groups.

In certain embodiments, the method includes, wherein the cell-encapsulating hydrogel components comprise: (a) a first backbone polymer comprising hyaluronic acid with an attached azide reactive group; (b) a second backbone polymer comprising heparin with an attached azide reactive group; (c) a cell attachment peptide comprising a cyclooctyne reactive group; and (d) a linking polymer comprising at least two cyclooctyne reactive groups.

In certain embodiments, the method includes, wherein the linking polymer comprises a polyethylene glycol (PEG) polymer, including where the linking polymer is a bifunctional PEG azide or a bifunctional PEG cyclooctyne.

In certain embodiments, the method includes, wherein the cell attachment peptide comprises an RGD tripeptide, including where the cell attachment peptide comprises the amino acid sequence GSGRGDSP (SEQ ID NO:1).

In certain embodiments, the method includes, wherein the cellularized hydrogel mixture comprises a concentration between 0.01 mM and 10 mM of the cell attachment peptide, including where the concentration of the cell attachment peptide is between 0.1 mM and 1.0 mM.

In certain embodiments, the method includes, wherein the cellularized hydrogel mixture comprises a weight-to-weight percentage of the second backbone polymer to the first backbone polymer between 0.01% and 0.15%, including where, wherein the weight-to-weight percentage of the second backbone polymer to the first backbone polymer between 0.03% and 0.10%.

In certain embodiments, the method includes, wherein the cell-encapsulating hydrogel components further comprise one or more pro-survival factors.

In certain embodiments, the method includes, wherein greater than 5% of the injected cells engraft into the affected area of the subject.

In certain embodiments, the method includes, wherein the cells of the cellular sample comprise neuronal cells, including where the neuronal cells are midbrain dopaminergic (mDA) neurons. In certain embodiments, the method includes, wherein the cells of the cellular sample comprise neuronal precursor cells, including where the neuronal precursor cells are midbrain dopaminergic (mDA) precursor cells.

In certain embodiments, the method includes, wherein the affected area of the subject is the subject's nervous system, including where the affected area of the subject is the subject's central nervous system and/or the subject's brain.

In certain embodiments, the method includes, wherein the cells of the cellularized hydrogel maintain a cellular phenotype in the affected area for at least one month (including at least four months) following the injection and/or at least 2% (including at least 5%) of the cells of the cellularized hydrogel maintain the cellular phenotype in the affected area for at least one month following the injection. In certain embodiments, the method includes, wherein the cellular phenotype comprises the expression of one or more cell type markers, including where the cellular phenotype comprises one or more cellular morphological characteristics or cell population morphological characteristics.

In certain embodiments, the method includes differentiating pluripotent progenitor cells into neuronal precursor cells or neuronal cells prior to the contacting, including where the differentiating comprises 2D or 3D cell culture.

In certain embodiments, the method includes, wherein the cell-encapsulating hydrogel components further comprise a dispersion factor, including where the dispersion factor promotes neurogenesis, neurite extension or a combination thereof and/or where the dispersion factor is selected from the group consisting of hepatocyte growth factor (HGF), glial derived neurotrophic factor (GDNF) and Ephrin-B2 (EFNB2). In certain embodiments, the dispersion factor is HGF and the cellularized hydrogel mixture comprises between 1 ng/ml and 100 ng/ml HGF, GDNF and the cellularized hydrogel mixture comprises between 1 ng/ml and 1 μg/ml GDNF or EFNB2 and the cellularized hydrogel mixture comprises between 0.1 ng/ml and 50 ng/ml EFNB2. In certain embodiments, the method includes, wherein the dispersion factor is encapsulated in the cellularized hydrogel, including where the dispersion factor is encapsulated in a controlled release system, or where the dispersion factor is covalently attached to one or more of the cell-encapsulating hydrogel components.

In certain embodiments, the method includes, wherein the dispersion factor comprises a cyclooctyne reactive group or an azide reactive group and the incubating comprises conditions sufficient to allow attachment of the dispersion factor to one or more of the cell-encapsulating hydrogel components.

In certain embodiments, the method includes, wherein the cellularized hydrogel has a gel stiffness that promotes dispersion of the cells of the cellular sample into the treatment site of the subject. In certain embodiments, the cellularized hydrogel is formulated with a weight-to-volume percentage of the first backbone polymer between 1% and 10%, including between 2% and 5%.

In certain embodiments, the method includes, wherein the cell-encapsulating hydrogel components comprise two or more dispersion factors, including where the two or more dispersion factors are selected from the group consisting of HGF, GDNF and EFNB2.

Aspects of the instant disclosure also include a cellularized hydrogel that includes: (a) a first backbone polymer comprising hyaluronic acid; (b) a second backbone polymer comprising heparin; (c) a linking polymer; (d) a dispersion factor; and (e) a plurality of cells responsive to the dispersion factor, wherein the first backbone polymer and the second backbone polymer are each linked to the linking polymer.

In certain embodiments, the cellularized hydrogel includes, wherein the first backbone polymer and the second backbone polymer are each linked to the linking polymer by a triazole moiety.

In certain embodiments, the cellularized hydrogel includes, wherein the dispersion factor is encapsulated in the cellularized hydrogel, including where the dispersion factor is encapsulated in a controlled release system.

In certain embodiments, the cellularized hydrogel includes, wherein the dispersion factor is covalently attached to one or more of the components of the cellular hydrogel, including where the dispersion factor is covalently attached by a triazole moiety. In certain embodiments, the dispersion factor is selected from the group consisting of hepatocyte growth factor (HGF), glial derived neurotrophic factor (GDNF) and Ephrin-B2 (EFNB2). In certain embodiments, the cellularized hydrogel includes two or more dispersion factors, including where the two or more dispersion factors are selected from the group consisting of HGF, GDNF and EFNB2. In certain embodiments, the cellularized hydrogel includes between 1 ng/ml and 100 ng/ml HGF, between 1 ng/ml and 1 μg/ml GDNF and/or between 0.1 ng/ml and 50 ng/ml EFNB2.

In certain embodiments, the cellularized hydrogel includes, wherein the dispersion factor promotes neurogenesis, neurite extension or a combination thereof.

In certain embodiments, the cellularized hydrogel includes, wherein the linking polymer comprises a polyethylene glycol (PEG) polymer, including a bifunctional PEG azide or a bifunctional PEG cyclooctyne.

In certain embodiments, the cellularized hydrogel includes a cell attachment peptide covalently attached to one or more component of the hydrogel, including covalently attached by a triazole moiety. In certain embodiments, the cell attachment peptide comprises an RGD tripeptide, including where the cell attachment peptide comprises the amino acid sequence GSGRGDSP (SEQ ID NO:1). In certain embodiments, the cellularized hydrogel includes a concentration between 0.01 mM and 10 mM, including between 0.1 mM and 1.0 mM, of the cell attachment peptide.

In certain embodiments, the cellularized hydrogel includes a weight-to-weight percentage of the second backbone polymer to the first backbone polymer between 0.01% and 0.15%, including between 0.03% and 0.10%.

In certain embodiments, the cellularized hydrogel includes one or more pro-survival factors.

In certain embodiments, the cellularized hydrogel includes, wherein the plurality of cells comprise neuronal cells, including where the neuronal cells comprise midbrain dopaminergic (mDA) neurons. In certain embodiments, the cellularized hydrogel includes, wherein the plurality of cells comprise neuronal precursor cells, including where the neuronal precursor cells comprise midbrain dopaminergic (mDA) precursor cells.

In certain embodiments, the cellularized hydrogel includes, wherein the cellularized hydrogel has a gel stiffness that promotes dispersion of the plurality of cells. In certain embodiments, the cellularized hydrogel is formulated with a weight-to-volume percentage of the first backbone polymer between 1% and 10%, including between 2% and 5%.

Aspects of the instant disclosure also include a kit for making a cellularized hydrogel that includes (a) a first backbone polymer comprising hyaluronic acid; (b) a second backbone polymer comprising heparin; (c) a linking polymer; and (d) a dispersion factor, wherein at least the linking polymer is in a separate container.

In certain embodiments, the kit includes a cell attachment peptide. In certain embodiments, the kit includes, wherein the first backbone polymer and the second backbone polymer each comprise an attached cyclooctyne reactive group and the linking polymer comprises at least two azide reactive groups. In certain embodiments, the kit includes, wherein the first backbone polymer and the second backbone polymer each comprise an attached azide reactive group and the linking polymer comprises at least two cyclooctyne reactive groups.

In certain embodiments, the kit includes, wherein the dispersion factor and/or the cell attachment peptide comprise an attached cyclooctyne reactive group or an attached azide reactive group.

In certain embodiments, the kit is configured for the preparation of a therapeutic cellularized hydrogel or the preparation of an in vitro tissue model cellularized hydrogel.

### BRIEF DESCRIPTION OF THE DRAWINGS

FIG. 1 provides a schematic representation of a cellularized hydrogel according to one embodiment of the instant disclosure.

FIG. 2A-2B demonstrates neural progenitor cell (NPC) differentiation into mature dopaminergic neurons within a synthetic 3D hyaluronic acid (HA) extracellular matrix (ECM) and the survival of transplanted Human Nuclear Antigen (HuNuc) expressing dopaminergic neurons encapsulated in three-dimensional (3D) HA hydrogels following transplantation into rats.

FIG. 3A-3D provides certain characteristics of HA hydrogels and the differentiation of midbrain dopaminergic (mDA) neurons according to embodiments of the instant disclosure.

FIG. 4A-4F demonstrates the effect of incorporating an RGD peptide and Heparin on mDA development and 3D neuronal cluster morphology according to certain embodiments of the instant disclosure.

FIG. 5A-5H provides certain characteristics of day 25 (D25) mDA neurons matured in 2D or 3D HA hydrogels according to certain embodiments of the instant disclosure.

FIG. 6A-6H provides certain characteristics of day 40 (D40) mDA neurons matured in 2D or 3D HA hydrogels according to certain embodiments of the instant disclosure.

FIG. 7 demonstrates the increased retention of live cells in a 3D platform as compared to a 2D platform according to one embodiment of the instant disclosure.

FIG. 8A-8F demonstrates the increase in vivo survival of mDA neurons transplanted with encapsulation within a 3D hydrogel of the instant disclosure as compared to survival without encapsulation.

FIG. 9 demonstrates the effects of RGD peptide and heparin functionalization of HA platforms on mDA neuronal maturation.

FIG. 10A-10C provides marker expression of mDA neuron progenitors generated on Mebiol gels for 15 days as described herein.

FIG. 11 demonstrates the dispersion promoting effects of certain functional factors according to embodiments of the instant disclosure.

FIG. 12 demonstrates the effect of different concentrations of certain functional factors on cell dispersion according to embodiments of the instant disclosure.

FIG. 13A-13H demonstrates the dispersion of midbrain dopaminergic (mDA) neurons cultured in 3D HA hydrogels and treated with various functional factors as described herein.

FIG. 14 demonstrates the effect of varied HA gel stiffness on cell dispersion according to embodiments of the instant disclosure.

FIG. 15 demonstrates the combined effects of increasing HA gel stiffness and the addition of certain functional factors on dispersion of populations of day (D25) mDA neurons.

FIG. 16A-16J depict dispersion of hESC-derived mDA neurons in three-dimensional (3D) HA hydrogels.

FIG. 17A-17C depict the effect of transplantation of hESC-derived mDA neurons co-encapsulated with dispersion factors on 6-OHDA unilesioned PD model rats.

FIG. 18A-18B depict the effect of 3DGF on motor function.

FIG. 19A-19H depict the effect of HA hydrogels with incorporated dispersion factors on survival and phenotype maintenance of co-transplanted hESC-derived mDA neurons at 20 weeks post translation.

FIG. 20A-20E depict enhanced synaptic integration and dispersion of hESC-derived mDA neurons transplanted with dispersive hydrogels, 20 weeks post-transplantation.

FIG. 21A-21B depict maintenance of mDA phenotype of hESC-derived mDA neurons transplanted with dispersive hydrogels, 20 weeks post-transplantation.

### DEFINITIONS

The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already afflicted (e.g., those with a neurological disorder) as well as those in which prevention is desired (e.g., those with increased susceptibility to a neurological disorder; those suspected of having a neurological disorder; those having one or more risk factors for a neurological disorder, etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as non-human primates, dogs, horses, cats, cows, sheep, goats, pigs, camels, etc. In some embodiments, the mammal is a human.

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated.

The terms “pluripotent cells”, “pluripotent stem cells”, “multipotent cells” and the like, as used herein refer to cells that are capable of differentiating into two of more different cell types and proliferating. Non limiting examples of pluripotent cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like. Pluripotent progenitor cells may be acquired from public or commercial sources or may be newly derived. As described herein, in some instances, pluripotent progenitor cells of the subject disclosure are those cells capable of giving rise to neuronal precursor cells and/or differentiated neurons. A cell may be naturally capable of giving rise to neuronal precursor cells and/or differentiated neurons or may be artificially made (e.g., reprogrammed, dedifferentiated, transdifferentiated, etc.) to be capable of giving rise to neuronal precursor cells and/or differentiated neurons. By “naturally capable” is meant that giving rise to neuronal precursor cells and/or differentiated neurons represents part of the natural developmental lineage or the natural differentiation potential of the cell. As such, cells made capable of giving rise to neuronal precursor cells and/or differentiated neurons artificially are generally cells that do not have such capability naturally.

The term “physiological conditions” is meant to encompass those conditions compatible with living cells, e.g., predominantly aqueous conditions of a temperature, pH, salinity, etc. that are compatible with living cells.

As used herein, the term “chemoselective functional group” refers to chemoselective reactive groups that selectively react with one another to form a covalent bond. Chemoselective functional groups of interest include, but are not limited to, two thiol groups, thiols and maleimide or iodoacetamide, as well as groups that can react with one another via Click chemistry, e.g., azide and alkyne groups (e.g., cyclooctyne groups). Chemoselective functional groups of interest, include, but are not limited to, thiols, alkyne, a cyclooctyne, an azide, a phosphine, a maleimide, an alkoxyamine, an aldehyde and protected versions thereof, and precursors thereof. In certain embodiments, the chemoselective functional group is a thiol.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the linker” includes reference to one or more linkers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The instant disclosure provides cellularized hydrogels containing cells encapsulated in linked polymers of hyaluronic acid, heparin and other components as described herein. Such cellularized hydrogels find use in variety of purposes including effective transplantation of cells into a host organism for cell therapy and the derivation of desired cell types. Other purposes include but are not limited to use as a tissue model for the in vitro study of cellular responses and behaviors. The instant disclosure also provides methods, including methods of making and using the described cellularized hydrogels. Also provided are kits that include components for making and/or using cellularized hydrogels e.g., according to the methods as described herein.

### Methods

The instant disclosure provides methods of making and/or using cellularized hydrogels as described herein. Hydrogels of the instant disclosure will generally be made by combining the components of the hydrogel, in liquid or solid/powder form, into a container or vessel, contacting the combined components with cells of a cellular sample and incubating such a mixture under conditions sufficient to allow formation of the hydrogel and encapsulation of the cells of the cellular sample within the hydrogel.

In some instances, methods of using a cellularized hydrogel may include preparing the hydrogel e.g., by mixing hydrogel components in an appropriate container or vessel, and adding cells to the hydrogel or otherwise contacting a cellular sample with one or more components of the hydrogel. As described in more detail elsewhere herein, components of the hydrogel may be functionally linkable including where two or more components of the hydrogel include compatible reactive groups or chemoselective functional groups that, when brought in sufficient proximity under appropriate conditions, are able to link the two components by a chemical bond, e.g., a covalent bond.

In some instances, components of the hydrogel may be combined into a hydrogel mixture where such a mixture may be defined as a combination of the components used to form the hydrogel prior to gelation. A cellularized hydrogel mixture will generally refer to a hydrogel mixture with cells added to the mixture but prior to gelation. In certain instances, a hydrogel mixture may be prepared by combining only those components that will not induce gelation when combined, mixing those components before or after adding the cells of a cellular sample to the mixture and subsequently adding the remaining components to the mixture that will induce gelation when combined. In some instances, the cells may be introduced as a cell pellet, e.g., as formed by centrifugation of suspended cells, and the cell pellet may be resuspended prior to or after adding one or more components that induce gelation but before gelation has occurred.

In many instances, a cellularized hydrogel mixture may be incubated under conditions sufficient for gelation of the hydrogel mixture and formation of the hydrogel. A cellularized hydrogel mixture may be incubated for a period of time sufficient for gelation and formation of the hydrogel. The conditions sufficient for gelation and/or the time period sufficient for gelation may vary depending e.g., on the particular components of the hydrogel, the particular relative concentrations of the hydrogel components, etc. For example, in some instances, the particular incubation conditions may be dependent on the cross-linking chemistry utilized in joining the components of the gel. In some instances, incubation of the cellularized hydrogel mixture may include a particular incubation temperature where such temperature may vary e.g., depending on the particular chemical reaction utilized in joining the hydrogel components, and may range from less than 4° C. to 42° C. or more including but not limited to e.g., 4° C. to 42° C., 4° C. to 37° C., 4° C. to 35° C., 4° C. to 30° C., 4° C. to 25° C., 4° C. to 20° C., 4° C. to 15° C., 4° C. to 10° C., 10° C. to 42° C., 15° C. to 42° C., 20° C. to 42° C., 25° C. to 42° C., 37° C. to 42° C., 10° C. to 37° C., 15° C. to 37° C., 20° C. to 37° C., 25 to 37° C., 30 to 37° C., 10° C. to 25° C., 15° C. to 25° C., 20° C. to 25° C., 4° C., 10° C., 15° C., 20° C., 21° C., 25° C., 30° C., 35° C., 37° C., 40° C., 42° C., and the like.

In some instances, incubation of the cellularized hydrogel mixture may or may not include external heating or cooling of the cellularized hydrogel mixture during the incubation. In some instances, incubation conditions and/or an incubation period sufficient for gelation may be those conditions sufficient for one or more chemical linking reaction(s) associated with reactive groups (e.g., chemoselective functional groups) present on one or more components of the hydrogel. For example, in some instances sufficient incubation conditions may include but are not limited to those reaction conditions sufficient for carrying out a “click chemistry” reaction including but not limited to an azide-alkyne cycloaddition reaction, a copper-catalyzed azide-alkyne cycloaddition (CuAAC) reaction, a strain-promoted azide-alkyne cycloaddition (SPAAC) reaction, and the like.

In some instances, incubation of the cellularized hydrogel mixture may be carried out under laboratory conditions, including e.g., at atmospheric pressure and at room temperature. In some instances, incubation of the cellularized hydrogel mixture may be carried out under cold-room conditions, including e.g., at atmospheric pressure and at below room temperature including e.g., at 4° C., at 10° C., at 15° C., etc. In other instances, incubation of the cellularized hydrogel mixture may be carried out at physiological conditions, including e.g., a human body temperature and at physiological pH.

Hydrogels of the subject disclosure may be introduced into a subject before, during or after gelation. In some instances, the hydrogel is allowed to gel before being introduced into a subject. In other instances, the hydrogel is not allowed to gel before being introduced into a subject.

Introduction of a cellularized hydrogel of the instant disclosure to a subject may serve various therapeutic purposes including providing the subject with an effective amount of cells for which the subject has a need and/or the subject is deficient in. In some instances, the instant disclosure includes a method of treating a mammalian subject, the method comprising a step of introducing a cellularized hydrogel of the present disclosure into the mammalian subject.

Cells, including those encapsulated in a cellular hydrogel, may be introduced by injection, catheter, intravenous perfusion, or the like depending on various factors including e.g., the viscosity of a cellular hydrogel as described herein. Cells may be frozen at liquid nitrogen temperatures and stored for long periods of time, being capable of use upon thawing. Once thawed, cells may be expanded by use of growth factors and/or feeder cells or in feeder-free conditions associated with e.g., pluripotent cell proliferation and differentiation. In some instances, cells may be administered fresh such that the cells are expanded and differentiated and administer without being frozen.

Introduction of cellularized hydrogel of the instant disclosure into a subject can be performed by a variety of means including but not limited to e.g., surgical implantation, injection, etc. For example, because the subject hydrogels have a reasonable working time (e.g., 5-10 minutes) prior to gelation the material can be injected through an appropriate needle which facilitates implantation including but not limited to e.g., an 18-28-gauge needle. A subject hydrogel, prior to gelation, can be injected into a mammalian subject in any convenient location or into any target tissue, depending on the desired outcome, including e.g., injection of the cellularized hydrogel into a target tissue where the target tissue is deficient in the cells encapsulated in the hydrogel.

Preparation of a cellularized hydrogel for injection may vary depending on the particular therapeutic context. In some instances, the hydrogel may be incubated in contact with the cells so as to encapsulate the cells during gelation the cell containing hydrogel may be loaded into an injector (including but not limited to e.g., a syringe, an injection “gun”, a needle or tubing affixed to a pump, etc.). Loading the cellularized gel may be performed by any convenient method including e.g., loading from the back of an injector where the “back” of the injector is defined as the end opposite the end from which the substance exits during injection.

In some embodiments, the physical properties of the hydrogel provide physical characteristics that decrease a cell's susceptibility to physical forces of injection that are detrimental to cell survival and/or delivery of viable cells. In some instances, the physical properties of the hydrogel protect the encapsulated cells from detrimental physical forces present during injection. Such physical forces that are detrimental to cell survival and/or the delivery of viable cells include but are not limited to e.g., shearing forces (e.g., shear stress from movement within a syringe and/or needle, hydrodynamic shear caused by shaking, etc.), compression forces, tension forces, etc.

Therapeutically effective amounts of the cells encapsulated within a hydrogel of the instant disclosure will vary depending e.g., on the condition to be treated, typical survival of the particular cell type within the host (e.g., including the average lifespan of cells of the particular cell type), etc.

In some embodiments, a therapeutically effective amount of cells is 1×10³ or more cells (including e.g., 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective amount of cells is in a range of from 1×10³ cells to 1×10¹⁰ cells (including e.g., from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁶ cells to 1×10¹⁰ cells, from 5×10⁶ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁹ cells, from 5×10⁵ cells to 5×10⁹ cells, from 1×10⁶ cells to 5×10⁹ cells, from 5×10⁶ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁶ cells to 1×10⁹ cells, from 5×10⁶ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁶ cells to 5×10⁸ cells, from 5×10⁶ cells to 5×10⁸ cells, from 1×10⁷ cells to 5×10⁸ cells, from 5×10⁷ cells to 5×10⁸ cells, or from 1×10⁸ cells to 5×10⁸ cells, etc.).

In some embodiments, the concentration of cells to be administered, e.g., in a therapeutically effective amount, is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/m1 to 1×10⁸ cells/ml, from 1×10⁶ cells/ml to 5×10⁷ cells/ml, from 1×10⁶ cells/ml to 1×10⁷ cells/ml, from 1×10⁶ cells/ml to 6×10⁶ cells/ml, or from 2×10⁶ cells/ml to 8×10⁶ cells/ml).

In some embodiments, the concentration of cells to be administered, e.g., in a therapeutically effective amount, is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁶ cells/ml or more, 4×10⁶ cells/ml or more, 5×10⁶ cells/ml or more, 6×10⁶ cells/ml or more, 7×10⁶ cells/ml or more, or 8×10⁶ cells/ml or more).

Encapsulated cells, as in a cellularized hydrogel of the instant disclosure, may be administered alone or may be co-administered in conjunction with other therapeutic agents, e.g., as a single co-therapy or as part of a treatment protocol or treatment schedule. When utilized, co-administered agents will vary depending on the particular condition to be treated and may include e.g., those agents conventionally administered as part of a therapy for the condition.

The terms “co-administration” and “in combination with” include the administration of two or more therapeutic agents (including a cellular therapeutic and a non-cellular therapeutic, two or more cellular therapeutics, etc.) either simultaneously, concurrently or sequentially within no specific time limits. In one embodiment, the agents are present in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Cellularized hydrogels of the instant disclosure may be utilized to introduce a therapeutically effective amount of a desired cell type to an affected area of a subject in need thereof. Such a subject will generally be those having a condition for which delivery of the desired cell type to the affected area will likely have some therapeutic benefit.

As such, subjects to which a cellularized hydrogel of the instant disclosure may be administered include but are not limited to e.g., a subject having a neurological disorder or a disease having neurological complications or otherwise resulting in a neuronal disorder including but not limited to e.g., Acquired Epileptiform Aphasia (Landau-Kleffner Syndrome), Adie's Pupil (Adie's Syndrome, Holmes-Adie syndrome), Agenesis of the Corpus Callosum, Agnosia, Aicardi Syndrome, Aicardi-Goutieres Syndrome Disorder (Cree encephalitis, Pseudo-Torch syndrome, Pseudotoxoplasmosis syndrome), Alexander Disease, Alpers' Disease (Progressive Sclerosing Poliodystrophy), Alzheimer's Disease, Amyotrophic Lateral Sclerosis (ALS), Anencephaly, Angelman Syndrome, Aphasia, Apraxia, Arachnoid Cysts (Intracranial Cysts), Arachnoiditis, Asperger Syndrome, Ataxia (Spinocerebellar Atrophy, Spinocerebellar Degeneration), Ataxia Telangiectasia, Attention Deficit-Hyperactivity Disorder, Autism Spectrum Disorder, Autonomic Dysfunction (Dysautonomia, Familial Dysautonomia, Riley-Day Syndrome), Batten Disease (Neuronal Ceroid Lipofuscinosis), Becker's Myotonia (Thomsen's Myotonia, Myotonia Congenita), Bell's Palsy, Benign Essential Blepharospasm (Blepharospasm), Benign Focal Amyotrophy (Hirayama Syndrome, O'Sullivan-McLeod Syndrome, Monomelic Amyotrophy), Benign Intracranial Hypertension (Pseudotumor Cerebri, Intracranial Hypertension), Bernhardt-Roth Syndrome (Lateral Femoral Cutaneous Nerve Entrapment, Meralgia Paresthetica), Binswanger's Disease (Subcortical Arteriosclerotic Encephalopathy), Bloch-Sulzberger Syndrome (Incontinentia Pigmenti), Brachial Plexus Birth Injuries (Erb-Duchenne Palsy, Dejerine-Klumpke Palsy), Brain Injury (Traumatic Brain Injury), Brain Tumors, Brown-Sequard Syndrome, Bulbospinal Muscular Atrophy (Kennedy's Disease, X-Linked Spinal and Bulbar Muscular Atrophy), Canavan Disease, Carpal Tunnel Syndrome, Causalgia (Complex Regional Pain Syndrome, Reflex Sympathetic Dystrophy Syndrome), Central Cervical Cord Syndrome, Central Pain Syndrome, Central Pontine Myelinolysis (Extrapontine Myelinolysis), Cephalic Disorders, Cerebellar Degeneration, Cerebellar Hypoplasia, Cerebral Atrophy, Cerebral Beriberi (Wernicke-Korsakoff Syndrome, Korsakoffs Amnesic Syndrome), Cerebral Gigantism (Sotos Syndrome), Cerebral Palsy, Cerebro-Oculo-Facio-Skeletal Syndrome (COFS) (Pena Shokeir II Syndrome, Cockayne Syndrome Type II), Charcot-Marie-Tooth Disease, Chiari Malformation, Chorea, Choreoacanthocytosis (Levine-Critchley Syndrome, Neuroacanthocytosis), Chronic Inflammatory Demyelinating Polyneuropathy (CIDP), Chronic Pain, Coffin Lowry Syndrome, Colpocephaly, Congenital Facial Diplegia (Moebius Syndrome), Corticobasal Degeneration, Craniosynostosis, Creutzfeldt-Jakob Disease, Dandy-Walker Syndrome, Dawson Disease (Subacute Sclerosing Panencephalitis), De Morsier's Syndrome (Septo-Optic Dysplasia), Dementia, Devic's Syndrome (Neuromyelitis optica (NMO)), Diabetic Neuropathy, Diffuse Sclerosis (Schilder's Disease), Dravet Syndrome, Dysgraphia, Dyslexia, Dysphagia, Dyspraxia, Dyssynergia Cerebellaris Myoclonica (Dyssynergia Cerebellaris Progressiva, Dentate Cerebellar Ataxia, Dentatorubral Atrophy, Primary Dentatum Atrophy, Ramsay Hunt Syndrome I), Dystonias, Encephalitis, Encephaloceles, Encephalopathy, Encephalotrigeminal Angiomatosis (Sturge-Weber Syndrome), Epilepsy, Erb's Palsy, Essential Tremor, Fahr's Syndrome (Familial Idiopathic Basal Ganglia Calcification), Fisher Syndrome (Miller Fisher Syndrome), Foot Drop, Friedreich's Ataxia, Frontotemporal Dementia, Gerstmann's Syndrome, Gerstmann-Straussler-Scheinker Disease, Giant Axonal Neuropathy, Glossopharyngeal Neuralgia, Guillain-Barré Syndrome, Hallervorden-Spatz Disease (NBIA), Head Injury, Hemicrania Continua, Hemifacial Spasm, Hemiplegia Alterans (Alternating Hemiplegia), Hereditary Neuropathies, Hereditary Spastic Paraplegia, Heredopathia Atactica Polyneuritiformis (Refsum Disease, Phytanic Acid Storage Disease), Holoprosencephaly, HTLV-1 Associated Myelopathy (Tropical Spastic Paraparesis), Huntington's Disease, Hydranencephaly, Hydromyelia (Syringohydromyelia), Hypertonia, Hypotonia, Immune-Mediated Encephalomyelitis (Postinfectious Encephalomyelitis, Acute Disseminated Encephalomyelitis), Infantile Neuroaxonal Dystrophy, Iniencephaly, Isaacs' Syndrome (Neuromyotonia), Joubert Syndrome, Kearns-Sayre Syndrome, Kleine-Levin Syndrome, Klippel-Feil Syndrome, Klüver-Bucy Syndrome, Kugelberg-Welander Disease (Spinal Muscular Atrophy), Kuru, Lambert-Eaton Myasthenic Syndrome, Lateral Medullary Syndrome (Wallenberg's Syndrome), Leigh's Disease, Lennox-Gastaut Syndrome, Lewy Body Dementia, Lipoid Proteinosis, Lissencephaly, Locked-In Syndrome, Lou Gehrig's Disease, Machado-Joseph Disease, Macrencephaly (Megalencephaly), Melkersson-Rosenthal Syndrome, Meningitis, Microcephaly, Migraine, Motor Neuron Diseases, Moyamoya Disease, Multifocal Motor Neuropathy, Multi-Infarct Dementia, Multiple Sclerosis, Multiple System Atrophy, Muscular Dystrophy, Myasthenia, Myoclonus, Myopathy, Myotonia, Narcolepsy, Neurofibromatosis, Neuroleptic Malignant Syndrome, Neurological Back Pain, Neurological Complications of AIDS, Neurological Complications of Lyme Disease, Neurological complications of Shingles, Neurological Complications of Tuberous Sclerosis, Neurological Consequences of Cytomegalovirus Infection, Neurological Deficiency Resulting from Aneurysm, Neurological Deficiency Resulting from Angiomatosis, Neurological Deficiency Resulting from Anoxia/Hypoxia, Neurological Deficiency Resulting from Arteriovenous Malformation, Neurological Deficiency Resulting from Atrial Fibrillation and Stroke, Neurological Deficiency Resulting from CADASIL, Neurological Manifestations of Pompe Disease, Neurological Sequelae Of Lupus, Neuronal Complications from a Leukodystrophy, Neuronal Migration Disorders, Neurosarcoidosis, Neurosyphilis, Neurotoxicity, Occipital Neuralgia, Ohtahara Syndrome, Olivopontocerebellar Atrophy, Opsoclonus Myoclonus, Paresthesia, Parkinson's Disease, Paroxysmal Choreoathetosis, Paroxysmal Hemicrania, Parry-Romberg, Pelizaeus-Merzbacher Disease, Perineural Cysts (Tarlov Cysts, Sacral Nerve Root Cysts), Peripheral Neuropathy, Periventricular Leukomalacia, Pervasive Developmental Disorders, Piriformis Syndrome, Pituitary Tumors, Polymyositis, Porencephaly, Primary Lateral Sclerosis, Progressive Locomotor Ataxia (Tabes Dorsalis, Syphilitic Spinal Sclerosis), Progressive Multifocal Leukoencephalopathy, Progressive Supranuclear Palsy (Steele-Richardson-Olszewski Syndrome), Ramsay Hunt Syndrome II, Rasmussen's Encephalitis, Repetitive Motion Disorders (Cumulative Trauma Disorders, Repetitive Stress Injuries, Overuse Syndrome), Restless Legs Syndrome, Rett Syndrome, Rheumatic Encephalitis, Schizencephaly, Shy-Drager Syndrome, Sotos Syndrome, Spasticity, Spina Bifida, Spinal Cord Infarction, Spinal Cord Injury, Spinal Tumors, Stiff-Person Syndrome, Striatonigral Degeneration, Stroke, Syringomyelia, Tardive Dyskinesia, Tethered Spinal Cord Syndrome, Todd's Paralysis, Tourette Syndrome, Transient Ischemic Attack (Mini Stroke), Transmissible Spongiform Encephalopathies, Transverse Myelitis, Tremor, Trigeminal Neuralgia, Troyer Syndrome, West Syndrome (Infantile Spasms), Williams Syndrome, etc.).

Subjects to which a cellularized hydrogel of the instant disclosure may be administered include but are not limited to e.g., a subject having a cancer (e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, etc.).

Subjects to which a cellularized hydrogel of the instant disclosure may be administered include but are not limited to e.g., a subject having an auto-immune disease (e.g., Diabetes Type I, Systemic Lupus, Sjogren's Syndrome, Myasthenia, Autoimmune Cytopenia, Scleromyxedema, Scleroderma, Crohn's Disease, Behcet's Disease, Rheumatoid Arthritis, Juvenile Arthritis, Multiple Sclerosis, Polychondritis, Systemic Vasculitis, Alopecia Universalis, Buerger's Disease, etc.),

Subjects to which a cellularized hydrogel of the instant disclosure may be administered include but are not limited to e.g., a subject having a cardiovascular disease (e.g., Acute Heart Damage, Chronic Coronary Artery Disease, etc.), a subject having an ocular disease (e.g., a subject in need of corneal regeneration, macular degeneration, etc.), a subject having an immune deficiency (e.g., Severe Combined Immunodeficiency Syndrome, X-linked Lymphoproliferative Syndrome, X-linked Hyper immunoglobulin M Syndrome, etc.).

Subjects to which a cellularized hydrogel of the instant disclosure may be administered include but are not limited to e.g., a subject having an anemia or other blood condition (e.g., Sickle Cell Anemia, Sideroblastic Anemia, Aplastic Anemia, Red Cell Aplasia, Amegakaryocytic Thrombocytopenia, Thalassemia, Primary Amyloidosis, Diamond Blackfan Anemia, Fanconi's Anemia, Chronic Epstein-Ban Infection, etc.).

Subjects to which a cellularized hydrogel of the instant disclosure may be administered include but are not limited to e.g., a subject having an acute wound or injury (e.g., Limb Gangrene, a subject in need of surface wound healing, a subject in need of jawbone replacement, a subject in need of skull bone repair, etc.), a subject having a metabolic disorder (e.g., Hurler's Syndrome, Osteogenesis Imperfecta, Krabbe Leukodystrophy, Osteopetrosis, Cerebral X-Linked Adrenoleukodystrophy, etc.), a subject having a liver disorder (e.g., Chronic Liver Failure, Liver Cirrhosis, etc.), a subject having a bladder disorder (e.g., Incontinence, End-Stage Bladder Disease, etc.), a subject having erectile dysfunction, etc.

Accordingly the affected areas to which a cellularized hydrogel of the instant disclosure may be administered may vary and may include but are not limited to e.g., an area of the nervous system (e.g., the central nervous system (CNS) (e.g., the brain or the spinal cord), an area of the peripheral nervous system (PNS)), an area of the cardiovascular system (e.g., the heart, the vascular system, etc.), an area of the skin, an area of an internal organ (e.g., the esophagus, the stomach, the intestine, the liver, the kidney, the bladder, etc.), and the like. In many instances, the affected area will be the primary area affected by the disorder being treated.

Methods of making and/or using a cellular hydrogel as described herein may also include one or more steps of culturing and/or differentiating or lineage restricting the cells to be encapsulated into the hydrogel prior to the encapsulation. Any convenient and appropriate method of cell culture and/or differentiation may find use performing such a step prior to contacting the cells with the hydrogel components including two-dimensional (2D) and three-dimensional (3D) methods of cell culture and differentiation. In some instances, pluripotent cells (e.g., stem cells of pluripotent progenitors) may be at least partially differentiated by 2D cell culture prior to encapsulation. In other instances, pluripotent cells (e.g., stem cells of pluripotent progenitors) may be at least partially differentiated by 3D cell culture prior to encapsulation.

In some instances, pluripotent progenitor cells may be at least partially differentiated into a neural lineage prior to encapsulation including but not limited to differentiated into NPCs (e.g., ESCs or iPS cell differentiated into NPCs), into neurons and/or neuronal cell types (e.g., sensory neurons, motor neurons, interneurons, unipolar neurons, bipolar neurons, multipolar neurons, anaxonic neurons, Basket cells, Betz cells, Lugaro cells, Medium spiny neurons, Purkinje cells, Pyramidal cells, Renshaw cells, Unipolar brush cells, Granule cells, Anterior horn cells, Spindle cells, Cholinergic neurons, GABAergic neurons, Glutamatergic neurons, Dopaminergic neurons, Serotonergic neurons, etc.), into a lineage restricted pluripotent cell type (e.g., mesenchymal stem cells, adipose stem cells, hematopoietic stem cells, etc.), into a terminally differentiated cell type, etc.

Suitable methods for differentiating pluripotent cells into neuronal cell types include but are not limited to e.g. culture on gels including e.g., those commercially available from Mebiol (Cosmobio, USA) including e.g., pNIPAAM-PEG 3D gels, or through the use of one or more differentiation media including e.g., neural differentiation medium, astrocyte differentiation medium, oligodendrocyte differentiation medium. Components of neural differentiation systems and/or mediums thereof may include or involve commercially available reagents from various manufactures including e.g., STEMCELL Technologies Inc. (Vancouver, BC); Thermo Fisher Scientific Inc. (Waltham, Mass.) and the like including one or more of the following reagents: STEMdiff™ Neural System, Dulbecco's Modified Eagle Medium (D-MEM), D-MEM/F-12 Medium, Dulbecco's Phosphate-Buffered Saline (D-PBS), StemPro® NSC SFM—Serum-Free Human Neural Stem Cell Culture Medium, N-2 Supplement, B-27® Serum-Free Supplement, Neurobasal® Medium, Fetal Bovine Serum, GlutaMAX™, Recombinant bFGF, Recombinant EGF, CTS™ CELLstart™ Substrate, Geltrex™ Reduced Growth Factor Basement Membrane Matrix, Poly-L-Ornithine, Laminin, Dibutyryl cAMP, Triiodo-L-Thyronine, matrigel, collagen matrix (e.g., 3D collagen matrix), collagen hydrogels, poly-L-lactide scaffolds, polyethylene glycol, polymer membranes (e.g., silicone polymer membranes), and the like.

In some instances, methods as described herein may include the derivation of pluripotent stem cells which may be encapsulated in a hydrogel matrix as described herein or may be partially or terminally differentiated prior to encapsulation. Methods of derivation of pluripotent cells from an autologous or non-autologous tissue useful in the methods described herein include but are not limited to, e.g., methods of embryonic stem cell derivation and methods of induced pluripotent stem cell derivation. In some instances, methods as described herein may be performed using non-autologous pluripotent cells previously derived including, e.g., those publically or available or commercially available (e.g., from Biotime, Inc., Alameda, Calif.). In some instances, methods as described herein may be performed using newly derived non-autologous pluripotent cells or newly derived autologous pluripotent cells including but not limited to, e.g., newly derived embryonic stem cells (ESC) (including, e.g., those derived under xeno-free conditions as described in, e.g., Lei et al. (2007) Cell Research, 17:682-688) and newly derived induced pluripotent stem cells (iPS). General methods of inducing pluripotency to derive pluripotent cells are described in, e.g., Rodolfa K T, (2008) Inducing pluripotency, StemBook, ed. The Stem Cell Research Community, doi/10.3824/stembook.1.22.1 and Selvaraj et al. (2010) Trends Biotechnol, 28(4)214-23, the disclosures of which are incorporated herein by reference. In some instances, pluripotent cells, e.g., iPS cells, useful in the methods described herein are derived by reprogramming and are genetically unmodified, including e.g., those derived by integration-free reprogramming methods, including but not limited to those described in Goh et al. (2013) PLoS ONE 8(11): e81622; Awe et al (2013) Stem Cell Research & Therapy, 4:87; Varga (2014) Exp Cell Res, 322(2)335-44; Jia et al. (2010) Nat Methods, 7(3):197-9; Fusaki et al. (2009) Proc Jpn Acad Ser B Phys Biol Sci. 85(8):348-62; Shao & Wu, (2010) Expert Opin Biol Ther. 10(2):231-42; the disclosures of which are incorporated herein by reference.

In some instances, methods of culturing cells, including e.g., pluripotent stem cells, may include xeno-free culture conditions wherein, e.g., human cells are not cultured with any reagents derived from non-human animals In some instances, methods culturing of pluripotent stem cells include feeder-free culture conditions, wherein the pluripotent stem cells are cultured under conditions that do not require feeder cells and/or in feeder cell free medium, including e.g., commercially available feeder-free mediums, such as, e.g., those available from STEMCELL Technologies, Inc. (Vancouver, BC). In some instances, methods culturing of pluripotent stem cells include culture conditions that include supplemental serum, including e.g. supplement of autologously derived serum, e.g., as described in Stute et al. (2004) Exp Hematol, 32(12):1212-25. In some instance the pluripotent cell media includes one or more pro-survival factors, e.g., including those described herein. General methods of culturing human pluripotent cells are described in, e.g., Freshney et al. (2007) Culture of human stem cells, Wiley-Interscience, Hoboken, N.J. and Borowski et al. (2012) Basic pluripotent stem cell culture protocols, StemBook, ed. The Stem Cell Research Community, StemBook, doi/10.3824/stembook, the disclosures of which are incorporated herein by reference.

Introduction of cells encapsulated in a hydrogel into a subject in need thereof (i.e., a host) as described herein will generally result in engraftment of the cells into the host. Levels of engraftment will vary depending e.g., on the particular cells introduced and/or the particular components and/or physical features of the hydrogel as provided herein. As such, in certain embodiments greater than 2% of the introduced cells will engraft into host tissue including but not limited to e.g., greater than 2% engraftment, greater than 3% engraftment, greater than 4% engraftment, greater than 5% engraftment, greater than 6% engraftment, greater than 7% engraftment, greater than 8% engraftment, greater than 9% engraftment, greater than 10% engraftment, greater than 11% engraftment, greater than 12% engraftment, greater than 13% engraftment, greater than 14% engraftment, greater than 15% engraftment, greater than 16% engraftment, greater than 17% engraftment, greater than 18% engraftment, greater than 19% engraftment, greater than 20% engraftment, etc. Engraftment may be measured by a variety of means including e.g., where the cells have a detectable marker that differentiates them from host tissue including e.g., a marker endogenous to the cells (e.g., an expressed antigen) or a marker heterologous (i.e., introduced) to the cells (e.g., a fluorescent dye or expressed fluorescent protein). In some instances, engraftment may be indirectly measured e.g., by measuring the presence of an identifying aspect of the introduced cells including e.g., an expressed marker or genetic mark (e.g., a distinct allele or gene) using any convenient and appropriate method including e.g., quantitative PCR, sequencing, etc.

Methods of the instant disclosure provide for long-term maintenance of engrafted cells within host tissue where the introduced cells remain viable and maintain a desired cell fate. Introduction of cells encapsulated in a hydrogel into a host as described herein will generally result in the engrafted cells maintaining a desired phenotype in the host where the phenotype may be indicative of the cell retaining a desired cell type. Levels of maintenance of a desired cell type will vary depending e.g., on the particular cells introduced and/or the particular components and/or physical features of the hydrogel as provided herein. As such, in certain embodiments, 1% or more of the introduced cells may maintain a particular desired cellular phenotype including but not limited to e.g., 2% or more, 3% or more, 4% or more, 5% or more, 6% or more, 7% or more, 8% or more, 9% or more, 10% or more, etc.

The length of time a cell may maintain a particular desired phenotype may likewise vary and as such cells introduced into a host using the hydrogels as described herein will persist within the target site of the host for a period of time ranging from days to multiple months or more including but not limited to e.g., 1 month or more, 1.5 months or more, 2 months or more, 2.5 months or more, 3 months or more, 3.5 months or more, 4 months or more, 4.5 months or more, 5 months or more, 5.5 months or more, 6 months or more, etc.

Desired phenotypes that may be maintained by cells introduced into a host using the hydrogel methods as describe herein will vary but will generally be those phenotypes indicative of desired cell viability, desired cell-type and/or desired cellular function. For example, in some instances, a cell introduced by use of a cellularized hydrogel may express and/or maintain one or more viability phenotypes (e.g., staining or non-staining with a viability indicating dye, movement/migration, viable morphology, etc.). In some instances, a cell introduced by use of a cellularized hydrogel may express and/or maintain one or more cell type markers (e.g., expression of a cell-surface cell type marker, expression of a cell type mRNA, etc.). Any convenient marker, e.g., for cell-type identification, may find use in determining a cellular phenotype of a cell including e.g., neuronal cell-type markers, stem cells or pluripotency markers, etc.

In some instances, suitable cell type markers include but are not limited to e.g., Microtubule-associated protein 2 (MAP2), Tyrosine Hydroxylase (TH), Forkhead Box A2 (FOXA2), LIM homeobox transcription factor 1 alpha (LMX1A), Msh Homeobox 1 (MSX1), Paired box 6 (PAX6), octamer-binding transcription factor 4 (OCT4) and Nanog homeobox (NANOG), Neuron-specific beta III Tubulin (Tuj1), Nuclear receptor related 1 protein (NURR1), dopamine active transporter (DAT), G protein-activated inward rectifier potassium channel 2 (GIRK2), Pituitary homeobox 3 (PITX3), Orthodenticle homeobox 2 (OTX2), engrailed homeobox 1 (EN1), etc., where a particular cell type may be indicated when by the presence or absence of one or a combination of markers.

In some instances, a cell introduced by use of a cellularized hydrogel may express and/or maintain one or more morphological characteristics indicative of a particular cell type including e.g., the extension of axons, the extension of dendrites, migration, shape, granularity, nuclear size, etc. In some instances, a morphological characteristic of a cell may be indicative of a desired cell function including but not limited to e.g., the extension of neurites (i.e., dendrites and/or axons). Other functional characteristics of cells may find use in measuring and/or identifying those cells having a desired phenotype including e.g., the measurement of action potentials generated by a desired neuronal cell type.

In some instances, the recovery or improvement in a particular depressed function or behavior of a subject may be indicative of sufficient engraftment and/or maintenance of a sufficient number of cells of a desired phenotype. Such improvements may, in some instances, be indicative of successfully treating the subject for the disorder.

In certain embodiments, before, during or after introduction of the cellularized hydrogel into the target area the hydrogel may be at least partially disintegrated e.g., to soften the cellularized hydrogel and/or dissolve the cellularized hydrogel. Any convenient method of at least partially disintegrated the cellularized hydrogel may find use in the described methods including but not limited to e.g., subjecting the cellularized hydrogel to one or more temperature changes (i.e., heating or cooling) to at least partially disintegrate the hydrogel (e.g., where the hydrogel is configured to be temperature sensitive), at least partially digesting the hydrogel with one or more enzymes, and the like.

As will be readily apparent, the methods as described above may make use of one or more the hydrogels and/or components thereof and/or kits containing such components as described below.

### Cellularized Hydrogels and Components Thereof

The instant disclosure includes cellularized hydrogels and components thereof for use in making a cellularized hydrogel and/or practicing the methods as described herein. Hydrogels of the instant disclosure will generally include one or more backbone polymers, and linking polymer and one or more functional factors. A cellularized hydrogel will generally include the components necessary for hydrogel formation and sufficient incubation to allow for encapsulation of cells within the hydrogel. A “hydrogel mixture”, as used herein will generally refer to the components of a hydrogel combined but prior to gelation, possibly but not necessarily excluding one or more components to prevent gelation, and may or may not be cellularized (i.e., may or may not contain cells).

The term “hydrogel” as used herein generally refers to a network of polymer chains (“hydrogel polymers”) that are water-insoluble, sometimes found as a colloidal gel in which water is the dispersion medium. Cellular hydrogel matrices can contain over 99% water and may comprise natural or synthetic polymers, or a combination thereof. As will be readily understood, a hydrogel need not contain 99% water, and may be configured to contain other percentages of water including e.g., where the components of the hydrogel are modified to generate particular physical properties including e.g., stiffness, density, etc. Hydrogels also possess a degree of flexibility due to their significant water content.

The stiffness module of a subject hydrogel can be in the range of from about 15 Pascals (Pa) to about 1500 Pa, e.g., from about 15 Pa to about 20 Pa, from about 20 Pa to about 50 Pa, from about 50 Pa to about 100 Pa, from about 100 Pa to about 150 Pa, from about 150 Pa to about 200 Pa, from about 200 Pa to about 250 Pa, from about 250 Pa to about 300 Pa, from about 300 Pa to about 350 Pa, from about 350 Pa to about 400 Pa, from about 400 Pa to about 500 Pa, from about 500 Pa to about 600 Pa, from about 600 Pa to about 700 Pa, from about 700 Pa to about 800 Pa, from about 800 Pa to about 900 Pa, from about 900 Pa to about 1000 Pa, from about 1000 Pa to about 1100 Pa, from about 1100 Pa to about 1200 Pa, from about 1200 Pa to about 1300 Pa, from about 1300 Pa to about 1400 Pa, or from about 1400 Pa to about 1500 Pa. Crosslinking of a particular gel may, in some instances be modulated (i.e., increased or decreased) to generate a stiffer or softer gel accordingly.

Various hydrogel polymers may find use in a particular hydrogel depending in part, e.g., on the end use of the hydrogel including e.g., what cell type is to be encapsulated in the hydrogel, into what target tissue the hydrogel is to be introduced, whether the hydrogel is to be used as an in vitro tissue model, etc. Hydrogel polymers suitable for use in various hydrogels include but are not limited to e.g., hydrogel polymers formed from the following monomers: lactic acid, glycolic acid, acrylic acid, 1-hydroxyethyl methacrylate (HEMA), ethyl methacrylate (EMA), propylene glycol methacrylate (PEMA), poly (N-isopropylacrylamide) (PNIPAAm), PNIPAAm-PEG, acrylamide (AAM), N-vinylpyrrolidone, methyl methacrylate (MMA), glycidyl methacrylate (GDMA), glycol methacrylate (GMA), ethylene glycol, fumaric acid, saccharides (e.g., monosaccharides (e.g., glucose (dextrose), fructose, galactose, ribose, glucuronic acid (e.g., D-glucuronic acid, β-D-glucuronic acid, etc.), acetylglucosamine (e.g., D-N-acetylglucosamine), iduronic acid (e.g., α-L-iduronic acid, 2-O-sulfo-α-L-iduronic acid, etc.) glucopyranosyls (e.g., 2-deoxy-2-acetamido-α-D-glucopyranosyl, 2-deoxy-2-sulfamido-α-D-glucopyranosyl, 2-deoxy-2-sulfamido-α-D-glucopyranosyl-6-O-sulfate, etc.), disaccharides (e.g., lactose, sucrose, glucuronate and N-acetylglucosamine disaccharides, etc.), polysaccharides, and the like.

In some instances, a hydrogel of the instant disclosure may include one or more hydrogel polymers where various hydrogel polymers may find use in the hydrogel polymers of the instant disclosure including but not limited to hyaluronic acid, heparin, polyethylene glycol (PEG), and the like.

Some or all components of the hydrogels as described herein will generally be “functionalized”, meaning the components will be modified with a functional group useful in forming the hydrogel and sufficiently encapsulating cells there within. As such components of the hydrogel may be modified with an attached chemoselective group and two or more components of the hydrogel may be modified with attached chemoselective groups that are compatible, i.e., compatible chemoselective groups, to facilitate joining of the components in a defined manner Useful chemoselective groups will vary depending on the particular context in which the hydrogel is used and, in some instances, the functionalized components may contain attached reactive groups that include but are not limited to e.g., those reactive groups compatible with click chemistry including but not limited to e.g., an azide reactive group, an alkyne reactive group, a cyclooctyne reactive group, a monofluorinated cyclooctyne reactive group, a difluorinated cyclooctyne reactive group, and the like. Useful cyclooctynes and cyclooctyne derivatives include but are not limited to e.g., aryl-less octynes, monofluorinated cyclooctynes, difluorinated cyclooctynes, dibenzocyclooctynes, biarylazacyclooctynones, dimethoxyazacyclooctynes, and the like. In some instances, two components joined by click chemistry or a cyclooctyne mediated reaction may be linked by a particular chemical moiety e.g., a heteroatom ring, a triazole moiety, and the like.

In particular embodiments described herein a first component of a hydrogel may be described as having a first member of a chemoselective group and a second hydrogel component may be described as having the second member of the chemoselective group. An ordinary skilled artisan will readily recognize that in many instances the chemoselective groups may be reversed or swapped such that the first component of the hydrogel may have the second member of the chemoselective group and the second component of the hydrogel may have the first component of the chemoselective group. Accordingly, in many embodiments, for simplicity only one arrangement of components and chemoselective groups is explicitly described. However, considering the ordinary artisan's understanding that such groups may be swapped, such embodiments should be considered as description with the chemoselective groups swapped where appropriate. As a non-limiting example, where a hyaluronic acid backbone polymer is described as having a cyclooctyne reactive group and a linking polymer is described as having an azide reactive group, an ordinary skilled artisan will readily understand that such reactive groups may be swapped such that the hydrogel components may instead include a hyaluronic acid backbone polymer having an azide reactive group and a linking polymer having a cyclooctyne reactive group.

In some instances, useful linking reactions may include other reactions useful in bioorthogonal chemistry including but not limited to e.g., Nitrone Dipole Cycloaddition, Norbornene Cycloaddition, Oxanorbornadiene Cycloaddition, Tetrazine Ligation, [4+1] Cycloaddition, Tetrazole Photoclick Chemistry, Quadricyclane Ligation, and the like.

In certain instances, a component of the hydrogel may include thiol functionalization including e.g., where the component may be referred to as having been “thiolated”. Any component of the hydrogel as described herein may be thiolated as desired and appropriate including but not limited to e.g., where the hydrogel includes thiolated heparin e.g., as described in Jha et al. Biomaterials. (2015) 47:1-12; PCT Publication No. WO2014/113573 and U.S. Patent Application Publication No. US 2015-0352156 A1, the disclosures of which are incorporated herein by reference in their entirety.

Certain components of a subject hydrogel need not be physically or covalently linked to other components of the hydrogel and may, in some instances, be associated with the hydrogel by simply mixing or incubating the component(s) with the hydrogel. In some instances, a component of a hydrogel may not be covalently linked to another component of the hydrogel but may instead by encapsulated within the hydrogel. In certain instances, components of a hydrogel of the instant disclosure may be associated through one or more noncovalent interactions including but not limited to e.g., electrostatic interactions, hydrogen bonding interactions, hydrophobic interactions, etc. In some instances, one or more functional factors may be associated with a hydrogel component, including but not limited to e.g., one or more backbone polymers, through one or more noncovalent interactions.

In some instances, all of the components of the hydrogel are physically linked together including e.g., where all components are covalently bound to one or more other components of the hydrogel.

### Backbone Polymers

Cellularized hydrogels of the instant disclosure will generally include one or more backbone polymers where a “backbone polymer” is a hydrogel polymer that provides primary structural support for the hydrogel. A backbone polymer, as described above, will generally include an attached reactive group for use in chemically linking the backbone polymer to other components of the hydrogel including e.g., another backbone polymer, a linking polymer, a functional factor, etc. Backbone polymers may vary in size and may range from an average molecular weight of 10 kDa or less to 500 kDa or more including but not limited to e.g., 10 kDa to 500 kDa, 10 kDa to 400 kDa, 10 kDa to 300 kDa, 10 kDa to 200 kDa, 10 kDa to 100 kDa, 25 kDa to 500 kDa, 25 kDa to 400 kDa, 25 kDa to 300 kDa, 25 kDa to 200 kDa, 25 kDa to 100 kDa, 50 kDa to 500 kDa, 50 kDa to 400 kDa, 50 kDa to 300 kDa, 50 kDa to 200 kDa, 50 kDa to 100 kDa, etc. In many instances, the backbone polymers of a subject hydrogel may have an average molecular weight of more than 10 kDa.

The backbone polymers of a cellularized hydrogel of the instant disclosure may be of a single type including where the cellularized hydrogel contains essentially one type of backbone polymer. In other instances, the backbone polymers of a cellularized hydrogel of the instant disclosure may be of a two or more different types including where the cellularized hydrogel contains essentially two different types of backbone polymer, three different types of backbone polymer, four different types of backbone polymer, five different types of backbone polymer, six different types of backbone polymer, seven different types of backbone polymer, eight different types of backbone polymer, nine different types of backbone polymer, ten different types of backbone polymer, etc.

In some instances, a hydrogel backbone of the instant disclosure may include at least two different hydrogel backbone polymers including e.g., at least a hyaluronic acid backbone polymer and a heparin backbone polymer.

Where a particular hydrogel composition includes at least two different backbone polymers such polymers may be combined in a particular ratio to produce desired hydrogel characteristics. For example, in some instances, a hydrogel having two different backbone polymers may have a weight-to-weight percentage of the second backbone polymer to the first backbone polymer ranging from less than 0.01% to 0.15% or more including but not limited to e.g., 0.01% to 0.15%, 0.01% to 0.14%, 0.01% to 0.13%, 0.01% to 0.12%, 0.01% to 0.11%, 0.01% to 0.10%, 0.01% to 0.09%, 0.01% to 0.08%, 0.01% to 0.07%, 0.01% to 0.05%, 0.02% to 0.15%, 0.03% to 0.15%, 0.04% to 0.15%, 0.05% to 0.15%, 0.06% to 0.15%, 0.07% to 0.15%, 0.08% to 0.15%, 0.09% to 0.15%, 0.10% to 0.15%, 0.02% to 0.10%, 0.03% to 0.10%, 0.04% to 0.10%, 0.05% to 0.10%, 0.06% to 0.10%, 0.04% to 0.08%, etc.

Whether a particular hydrogel contains a single backbone polymer or multiple backbone polymers, including two or more backbone polymers, the hydrogel may contain a particular amount of the primary backbone polymer where such amount provides suitable physical properties in the hydrogel following gelation, including e.g., stiffness, density, etc. In some instances, a suitable weight-to-volume percentage of the primary backbone polymer may range from less than 1% to more than 10% including but not limited to e.g., 1%, 1.5%, 2%, 2.5%, 3%, 3.5%, 4%, 4.5%, 5%, 5.5%, 6%, 6.5% 7%, 7.5%, 8%, 8.5%, 9%, 9.5%, 10%, 1% to 10%, 2% to 10%, 3% to 10%, 4% to 10%, 5% to 10%, 6% to 10%, 7% to 10%, 8% to 10%, 9% to 10%, 1% to 9%, 2% to 9%, 3% to 9%, 4% to 9%, 5% to 9%, 6% to 9%, 7% to 9%, 8% to 9%, 1% to 8%, 2% to 8%, 3% to 8%, 4% to 8%, 5% to 8%, 6% to 8%, 7% to 8%, 1% to 7%, 2% to 7%, 3% to 7%, 4% to 7%, 5% to 7%, 6% to 7%, 1% to 6%, 2% to 6%, 3% to 6%, 4% to 6%, 5% to 6%, 1% to 5%, 2% to 5%, 3% to 5%, 4% to 5%, etc.

In some instances, a backbone polymer may serve one or more additional functions beyond providing structural support for a cellularized hydrogel of the instant disclosure including but not limited to e.g., where one or more of the backbone polymers promote differentiation of the encapsulated cells to a desired cell type, promote maintenance of a desired phenotype in the encapsulated cells, promotes viability of the encapsulated cells, etc.

### Linking Polymers

Cellularized hydrogels of the instant disclosure will generally include one or more linking polymers where a “linking polymer” is a hydrogel polymer that provides primary linkage between components, including e.g., backbone polymers, of the hydrogel. A linking polymer, as described above, will generally include an attached reactive group that is compatible with or complementary to one or more reactive groups present on other components of the hydrogel including, e.g., on one or more backbone polymers, for use in chemically linking or crosslinking the hydrogel Linking polymers may vary in size and may range from an average molecular weight of 1 kDa or less to 50 kDa or more including but not limited to e.g., 1 kDa to 50 kDa, 1 kDa to 40 kDa, 1 kDa to 30 kDa, 1 kDa to 20 kDa, 1 kDa to 10 kDa, 2 kDa to 30 kDa, 2 kDa to 20 kDa, 2 kDa to 10 kDa, 5 kDa to 50 kDa, 5 kDa to 40 kDa, 5 kDa to 30 kDa, 5 kDa to 20 kDa, 5 kDa to 10 kDa, etc. In many instances, the backbone polymers of a subject hydrogel may have an average molecular weight of 10 kDa or less.

The linking polymers of a cellularized hydrogel of the instant disclosure may be of a single type including where the cellularized hydrogel contains essentially one type of linking polymer. In other instances, the linking polymers of a cellularized hydrogel of the instant disclosure may be of a two or more different types including where the cellularized hydrogel contains essentially two different types of linking polymer, three different types of linking polymer, four different types of linking polymer, five different types of linking polymer, etc.

Any convenient and appropriate polymer may find use as a linking polymer in a cellular hydrogel of the instant disclosure including naturally occurring polymers and synthetic polymers including but not limited to e.g., peptides, glycol polymers, collagen polymers, cellulose polymers, latex polymers, etc. In some instances, a linking polymer may be a polyethylene glycol (PEG) polymer including e.g., monofunctional PEG polymers, bifunctional PEG polymers including e.g., homobifunctional PEG polymers and heterobifunctional PEG polymers. Useful PEG polymers may include but are not limited to e.g., Azide (—N₃) Heterobifunctional Functionalized PEG, Dibenzylcyclooctyne (DBCO) Heterobifunctional Functionalized PEG, Biotin Heterobifunctional Functionalized PEG, Maleimide Heterobifunctional Functionalized PEG, NHS Ester Heterobifunctional Functionalized PEG, Thiol Heterobifunctional Functionalized PEG, COOH Heterobifunctional Functionalized PEG, Amine Heterobifunctional Functionalized PEG, Hydroxyl Heterobifunctional Functionalized PEG, Acrylate/Methacrylate Heterobifunctional Functionalized PEG, 2-Bromoisobutyrate Mono- or Homobifunctional Functionalized PEG, ATRP Mono- or Homobifunctional Functionalized PEG, Acetylene Mono- or Homobifunctional Functionalized PEG, Acrylamide Mono- or Homobifunctional Functionalized PEG, Acrylate Mono- or Homobifunctional Functionalized PEG, Alkyl Mono- or Homobifunctional Functionalized PEG, Amine Mono- or Homobifunctional Functionalized PEG, Azide Mono- or Homobifunctional Functionalized PEG, Dibenzylcyclooctyne (DBCO) Mono- or Homobifunctional Functionalized PEG, Bromide Mono- or Homobifunctional Functionalized PEG, Carboxylic Acid Mono- or Homobifunctional Functionalized PEG, Chloroformate Mono- or Homobifunctional Functionalized PEG, Epoxide Mono- or Homobifunctional Functionalized PEG, Hydroxyl Mono- or Homobifunctional Functionalized PEG, Methacrylate Mono- or Homobifunctional Functionalized PEG, NHS Ester Mono- or Homobifunctional Functionalized PEG, Propionic Acid Mono- or Homobifunctional Functionalized PEG, RAFT Mono- or Homobifunctional Functionalized PEG, Thiol Mono- or Homobifunctional Functionalized PEG, Tosylate Mono- or Homobifunctional Functionalized PEG, Vinyl ether Mono- or Homobifunctional Functionalized PEG, Vinylsulfone Mono- or Homobifunctional Functionalized PEG, bis-MPA dendron Mono- or Homobifunctional Functionalized PEG, PEG Dendrimers, multi-branched PEG polymers and the like. PEG polymers of the instant disclosure may be mono- or bifunctional, including heterobifunctional or homobifunctional, with any convenient reactive group including but not limited to those reactive groups described herein.

In many embodiments, linking polymers of the subject disclosure may be bifunctional, i.e., include two reactive groups where such reactive groups will be the same or different. Bifunctional linking polymers may facilitate the crosslinking of two molecules or components of the hydrogel including e.g., linking two backbone polymers, linking a backbone polymer and a functional factor, etc.

### Functional Factors

Cellularized hydrogels of the instant disclosure may include one or more functional factors. The term “functional factors” as described herein include those hydrogel components that functionally promote the survival of encapsulated cells, modify or maintain a phenotype of the encapsulated cells, modify or induce a behavior of the encapsulated cells, etc. The function of functional factors may be determined by a variety of means including e.g., culturing a population of cells with and without the subject functional factor and comparing the cellular function, viability, phenotype or behavior of the cultured cells.

Functional factors of the subject hydrogels may or may not be covalently attached to one or more other components of the hydrogel. For example, in some instances, one or more functional factors are covalently attached to a component of the hydrogel including but not limited to e.g., a backbone polymer of the hydrogel. In other instances, one or more functional factors are noncovalently attached to a component of the hydrogel including but not limited to e.g., a backbone polymer of the hydrogel.

Without being bound by theory, in some instances, the functional properties of heparin may facilitate noncovalent association of a functional factor as described herein with the hydrogel. For example, in some instances, heparin, covalently linked to a backbone polymer of the hydrogel including but not limited to e.g., hyaluronic acid, facilitates association of one or more functional factors with the hydrogel through noncovalent interactions between the heparin and the one or more functional factors. As such, in certain instances, the functional factors of the instant disclosure may include those having an overall basic isoelectric point (i.e., an isoelectric point of greater than 7.0 including but not limited e.g., an isoelectric point of 7.0 to 11.0, 7.0 to 10.0, 7.0 to 9.0, 7.0 to 8.0, 8.0 to 11.0, 8.0 to 10.0, 8.0 to 9.0, 9.0 to 11.0, 9.0 to 10.0, etc.). In certain instances, the functional factors of the instant disclosure may include those having a heparin binding domain including but not limited to e.g., those described in Murioz & Linhardt Arteriosclerosis, Thrombosis, and Vascular Biology. (2004) 24:1549-1557; Perkins et al. Front Immunol. (2014) 5:126; Wu et al. Blood Coagul Fibrinolysis. (1994) 5(1):83-95, the disclosures of which are incorporated herein by reference in their entirety.

In some instances, functional factors of cellularized hydrogel may include dispersion factors. The term “dispersion factors” will generally include those factors that promote the migration of the encapsulated cells out of the hydrogel. Dispersion factors may be general dispersion factors that function on many or all cell types capable of dispersion or may be specific dispersion factors that function on one or a small number of closely related cell types capable of dispersion. For example, in some instances, dispersion factors may be specific for the dispersion of neurons and/or neural progenitors and may be referred to as neural dispersion factors. Neural dispersion factors may or may not have functions beyond inducing or promoting cell dispersion including but not limited to e.g., promoting neurogenesis, promoting neurite extension, etc.

Cellular dispersion may be evaluated by any convenient method including those conventionally used to evaluate cell migration including timepoint, time-lapse and video imaging. In some instances, cellular dispersion may quantified by measuring internuclear distances of cells at two or more time points where the nuclei of cells may be identified by the use of a nuclear marker including but not limited to a fluorescent nuclear marker including e.g., fluorescent nucleic acid probes including DAPI, Hoechst Dyes, PicoGreen, RiboGreen, OliGreen, cyanine dyes (e.g., YO-YO), ethidium bromide, SybrGreen, DRAQ dyes (e.g., DRAQ5, DRAQ7), and the like. Internuclear distances may, in some instances, be measured through the use of automated image analysis software.

In certain embodiments, useful dispersion factors may include but are not limited to e.g., growth factors (e.g., hepatocyte growth factor (HGF), fibroblast growth factor (FGF), etc.), neurotrophic factors (e.g., glial derived neurotrophic factor (GDNF)), Ephrins and associated receptors (e.g., Ephrin-B1 (EFNB1), Ephrin-B2 (EFNB2), etc.), BMP family members, Contactins, ECM molecules, Hedgehog family members, Nectins, Netrins (e.g., Netrin-1) and associated receptors, Nogo proteins and associated receptors, Repulsive Guidance Molecules and Receptors (e.g., DAPK3/ZIPK, DCC, FAK, LARG, Neogenin, RGM-A, RGM-B, RGM-C/Hemojuvelin, ROCK1, ROCK2, SHP/NROB2, SHP-1, SHP-2, Src, UNC5H1, UNC5H3, UNC5H4, UNC5H2/UNC5B, etc.), Semaphorins, Plexin Receptors, Slit Ligands and ROBO Receptors SLITRK Family proteins, Wnt Family proteins, Other Axon Guidance Proteins (e.g., Cadherin-10, Caldesmon/CALD1, CDK5 Activator 1, DOCK3, Draxin, DSCAM, EMP3, Endophilin A1/SH3GL2, F-Spondin/SPON1, GAP-43, GPR6, Katanin p60, Kilon/NEGR1, Lamin B1, LINGO-1, LINGO-3, LINGO-4, LRRN1/NLRR-1, LRRN3/NLRR-3, MDGA1, MDGA2, Mena, Neurocan, Neurofascin, Ninjurin-1, Ninjurin-2, PRG-1/LPPR4, Stathmin-2/STMN2, VASP, and the like.

In some instances, one or more dispersion factors may be associated with the hydrogel by a noncovalent interaction with a component of the hydrogel. In other instances, one or more dispersion factors may be covalently bound to another component of the hydrogel.

Effective amounts of the subject dispersion factors will vary depending on e.g., the specific types of cells used. In certain embodiments an effective amount of HGF may range from less than 1 ng/ml to more than 100 ng/ml HGF including but not limited to e.g., 1 ng/ml to 100 ng/ml, 1 ng/ml to 80 ng/ml, 1 ng/ml to 60 ng/ml, 1 ng/ml to 40 ng/ml, 1 ng/ml to 30 ng/ml, 1 ng/ml to 20 ng/ml, 2 ng/ml to 100 ng/ml, 2 ng/ml to 80 ng/ml, 2 ng/ml to 60 ng/ml, 2 ng/ml to 40 ng/ml, 2 ng/ml to 30 ng/ml, 2 ng/ml to 20 ng/ml, 3 ng/ml to 100 ng/ml, 3 ng/ml to 80 ng/ml, 3 ng/ml to 60 ng/ml, 3 ng/ml to 40 ng/ml, 3 ng/ml to 30 ng/ml, 3 ng/ml to 20 ng/ml, 1 ng/ml to 10 ng/ml, 1 ng/ml to 5 ng/ml, 10 ng/ml to 30 ng/ml, etc.

Effective amounts of the subject dispersion factors will vary depending on e.g., the specific types of cells used. In certain embodiments an effective amount of GDNF may range from less than 1 ng/ml to more than 1000 ng/ml GDNF including but not limited to e.g., 1 ng/ml to 1000 ng/ml, 1 ng/ml to 800 ng/ml, 1 ng/ml to 600 ng/ml, 1 ng/ml to 400 ng/ml, 1 ng/ml to 300 ng/ml, 1 ng/ml to 200 ng/ml, 1 ng/ml to 100 ng/ml, 10 ng/ml to 1000 ng/ml, 10 ng/ml to 800 ng/ml, 10 ng/ml to 600 ng/ml, 10 ng/ml to 400 ng/ml, 10 ng/ml to 300 ng/ml, 10 ng/ml to 200 ng/ml, 10 ng/ml to 100 ng/ml, 100 ng/ml to 1000 ng/ml, 100 ng/ml to 800 ng/ml, 100 ng/ml to 600 ng/ml, 100 ng/ml to 400 ng/ml, 100 ng/ml to 300 ng/ml, 100 ng/ml to 200 ng/ml, 10 ng/ml to 30 ng/ml, 50 ng/ml to 150 ng/ml, 400 ng/ml to 600 ng/ml, etc.

Effective amounts of the subject dispersion factors will vary depending on e.g., the specific types of cells used. In certain embodiments an effective amount of EFNB2 may range from less than 0.1 ng/ml to more than 100 ng/ml EFNB2 including but not limited to e.g., 0.1 ng/ml to 100 ng/ml, 0.1 ng/ml to 80 ng/ml, 0.1 ng/ml to 60 ng/ml, 0.1 ng/ml to 40 ng/ml, 0.1 ng/ml to 30 ng/ml, 0.1 ng/ml to 20 ng/ml, 0.1 ng/ml to 10 ng/ml, 1 ng/ml to 100 ng/ml, 1 ng/ml to 80 ng/ml, 1 ng/ml to 60 ng/ml, 1 ng/ml to 40 ng/ml, 1 ng/ml to 30 ng/ml, 1 ng/ml to 20 ng/ml, 1 ng/ml to 10 ng/ml, 2 ng/ml to 100 ng/ml, 2 ng/ml to 80 ng/ml, 2 ng/ml to 60 ng/ml, 2 ng/ml to 40 ng/ml, 2 ng/ml to 30 ng/ml, 2 ng/ml to 20 ng/ml, 2 ng/ml to 10 ng/ml, 1 ng/ml to 5 ng/ml, 1 ng/ml to 3 ng/ml, 5 ng/ml to 15 ng/ml, etc.

Dispersion factors may or may not be physically or covalently attached to components of the hydrogel. For example, in other instances, one or more dispersion factors may be covalently attached to one or more components of the hydrogel including e.g., one or more backbone polymers, one or more linking polymers, etc., though the use of a functional group as described herein. In other instances a dispersion factor is not physically attached to the hydrogel but instead mixed or associated with the hydrogel. Non-physically attached dispersion factors may in some instances, be provided associated with or encapsulated in the hydrogel in a controlled release form (e.g., as part of a controlled release system).

In some instances, two or more different dispersion factors may be employed, including where the different dispersion factors are covalently attached to components of the hydrogel or one or more, including all, the components are not covalently attached to components of the hydrogel. Useful numbers of different dispersion factors include but are not limited to e.g., one or more, two or more, three or more, four or more, five or more, etc.

Hydrogels of the instant disclosure may also include pro-survival factors. Pro-survival factors, as used herein, generally include those factors that promote at least one of cell viability and cell proliferation and may include e.g., growth factors and other pro-survival factors including but not limited to, e.g., Rho-associated kinase (ROCK) inhibitor, pinacidil, allopurinol, uricase, cyclosporine, ZVAD-fmk, pro-survival cytokines (e.g., insulin-like growth factor-1 (IGF-1)), Thiazovivin, etc. In some instances, one or more pro-survival factors may be associated with the hydrogel by a noncovalent interaction with a component of the hydrogel. In other instances, one or more pro-survival factors may be covalently bound to another component of the hydrogel.

In some instances, a subject hydrogel may include one or more factors that promote the growth, proliferation, survival, differentiation (e.g., a factor that promotes differentiation; a factor that inhibits differentiation; a factor that reverses differentiation, e.g., a de-differentiation factor, a pluripotency factor, etc.; and the like), and/or function of a cell encapsulated by the hydrogel.

As used herein, the term “growth factor” is used broadly to encompass factors that modulate the growth, proliferation, survival, differentiation, and/or function of a cell. Suitable growth factors include, but are not limited to: a colony stimulating factor (e.g., Neupogen® (filgrastim, G-CSF), Neulasta (pegfilgrastim), granulocyte colony stimulating factor (G-CSF), granulocyte-monocyte colony stimulating factor, macrophage colony stimulating factor, megakaryocyte colony stimulating factor, and the like), a growth hormone (e.g., a somatotropin, e.g., Genotropin®, Nutropin®, Norditropin®, Saizen®, Serostim®, Humatrope®, a human growth hormone, and the like), an interleukin (e.g., IL-1, IL-2, including, e.g., Proleukin®, IL-3, IL-4, IL-5, IL-6, IL-7, IL-8, IL-9, etc.), a growth factor (e.g., Regranex® (beclapermin, PDGF), Fiblast® (trafermin, bFGF), Stemgen® (ancestim, stem cell factor), keratinocyte growth factor, an acidic fibroblast growth factor, a stem cell factor, a basic fibroblast growth factor, and the like), a chemokine (e.g., IP-10, Mig, Groa/IL-8, RANTES, MIP-la, MIP-1β, MCP-1, PF-4, and the like), an angiogenic agent (e.g., vascular endothelial growth factor (VEGF)), an EGF (epidermal growth factor), a receptor tyrosine kinase ligand, thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, a tissue factor, an insulin-like growth factor, a luteinizing hormone, a follicle stimulating hormone, a macrophage activating factor, tumor necrosis factor, a neutrophil chemotactic factor, a nerve growth factor, a tissue inhibitor of metalloproteinases, a vasoactive intestinal peptide, angiogenin, angiotropin, fibrin, hirudin, a leukemia inhibitory factor, a Wnt signaling ligand (e.g., Wnt, norrin, R-spondin, etc.), a Wnt signaling inhibitor (e.g., WIF (Wnt inhibitory factor), sFRP (Secreted Frizzled Related Protein), Dkk (Dickkopf), Notum, and the like), a Notch or Notch ligand protein, a receptor tyrosine kinase ligand, a hedgehog (HH) pathway ligand (e.g., HH), and a transforming growth factor-β (TGF-β). In certain embodiments, the factor is a cell growth factor. In specific embodiments, the factor is a growth factor that promotes the growth of a neural progenitor cell. In some instances, one or more growth factors may be associated with the hydrogel by a noncovalent interaction with a component of the hydrogel. In other instances, one or more growth factors may be covalently bound to another component of the hydrogel.

Hydrogels of the instant disclosure may include one or more cell attachment peptides, also referred to herein as “cell-adhesive peptides”, where such attachment peptides generally serve to promote attachment of the encapsulated cells to one or more components of the hydrogel. Cell attachment peptides may also, in some instances, promote viability and/or other cellular functions or behaviors. Any convenient cell attachment peptide may find use in the subject hydrogels including but not limited to e.g., those containing an RGD tripeptide including but not limited to e.g., a peptide containing the amino acid sequence GSGRGDSP (SEQ ID NO:1). Effective concentrations of cell attachment peptides may vary depending e.g., on the cell type encapsulated in the hydrogel and may range in concentration from less than 0.01 mM to 10 mM or more including but not limited to e.g., 0.01 mM, 0.05 mM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 0.01 mM to 10 mM, 0.01 mM to 1 mM, 0.1 mM to 10 mM, 0.1 mM to 1 mM, etc.

In some instances, cell attachment peptides of the instant disclosure may also include but are not limited to e.g., collagen, collagen-mimetic peptides (e.g., peptides containing a GFOGER (SEQ ID NO:3; where 0=hydroxyproline) sequence or GFOGER-like sequence as described in e.g., Wojtowicz et al. Biomaterials. 2010 March; 31(9):2574-82 and Zhang et al. J Biol Chem. (2003) 278(9):7270-7, the disclosures of which are incorporated herein by reference in their entirety), laminin derived and biomimetic peptides (including but not limited to e.g., YIGSR (SEQ ID NO:4) containing peptides (as described in e.g., Boateng et al. Am J Physiol Cell Physiol. (2005) 288(1):C30-8 and Yoshida et al. Br J Cancer. (1999) 80(12):1898-904, the disclosures of which are incorporated herein by reference in their entirety), IKVAV (SEQ ID NO:5) containing peptides (as described in e.g., Yamada et al. FEBS Lett. (2002) 530(1-3):48-52 and Tashiro et al. JCB (1989) 264(27)16174-82; the disclosures of which are incorporated herein by reference in their entirety), PDSGR (SEQ ID NO:6) containing peptides (as described in e.g., Maeda et al. Biochem Biophys Res Commun. 1998 Jul. 30; 248(3):485-9; the disclosure of which is incorporated herein by reference in its entirety), etc.); bone sialoprotein derived peptides (e.g., peptides containing a GGGNGEPRGDTYRAY (SEQ ID NO:7) as described in e.g., Rezania et al. J Biomed Mater Res. (1997) 37(1):9-19, the disclosure of which is incorporated herein by reference in its entirety), fibronectin derived and biomimetic peptides (including but not limited to e.g., PHSRN (SEQ ID NO:8) containing peptides (as described in e.g., Feng & Mrksich. Biochemistry. (2004) 43(50):15811-2 and Livant et al. J Clin Invest. (2000) 105(11): 1537-1545; the disclosures of which are incorporated herein by reference in their entirety), REDV (SEQ ID NO:9) containing peptides (as described in e.g., Wang et al. J Biomed Mater Res A. (2015) 103(5):1703-12 and Ji et al. J Biomed Mater Res A. (2012) 100(6):1387-97; the disclosures of which are incorporated herein by reference in their entirety), LDV containing peptides (as described in e.g., Wayner & Kovach J Cell Biol. (1992) 116(2):489-97; the disclosure of which is incorporated herein by reference in its entirety), GRGDSP (SEQ ID NO:10) containing peptides (as described in e.g., Patel et al. J Biomed Mater Res A. (2007) 83(2):423-33; the disclosure of which is incorporated herein by reference in its entirety), etc.); placenta growth factor derived and biomimetic peptides (including but not limited to e.g., P1GF-2(123-144) preptides as described in e.g., Martino et al. Science. (2014) 343(6173):885-8; the disclosure of which is incorporated herein by reference in its entirety), and the like.

In certain embodiments, the cell attachment peptide has a length of 40 amino acids or less, 35 amino acids or less, 30 amino acids or less, 25 amino acids or less, or 20, 19, 18, 17, 16, 15, 14, 13, 12, 11, 10, 9, 8, 7, 6, or 5 amino acids or less. For example, in some cases, the cell attachment peptide has a length of from about 3 amino acids to about 40 amino acids, e.g., from about 3 amino acids to about 5 amino acids, from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, or from about 35 amino acids to about 40 amino acids.

In certain instances, a functional factor, as described herein, may serve multiple, including dual, purposes. For example, in some instances, a functional factor may be a dual-purpose functional factor and may e.g., function as both a dispersion factor and a pro-survival factor.

The function of a particular functional factor (including whether the factor has a single or multiple functions) may, in certain circumstances, be dependent on the cells of the cellularized hydrogel and the particular response, or responses, of such cells to the functional factor. For example, in some instances, a the cells of a cellularized hydrogel may respond to a particular growth factor with dispersion and thus the particular growth factor may serve as a dispersion factor for the particular cell type. However, a different cell type may respond to the same particular growth factor differently, e.g., without dispersion and with an increase in survival, proliferation or the like and, as such, in relationship to the second cell type the particular growth factor may serve not as a dispersion factor but instead as a pro-survival factor. As such, the particular function of a functional factor may, in some instances, depend on the cells to which the factor is introduced. In certain instances, a particular functional factor may have essentially the same function across various cell types and, as such, may generally function as a particular functional factor type (i.e., a pro-survival factor, a dispersion factor, etc.) across all relevant cell types.

The predicted functional response(s) of a particular cell type to a particular functional factor may be inferred, in certain cases, from previously attributed in vitro or in vivo roles of the factor in cell behaviors of the cell type e.g., as previously described in the scientific literature. In some instances, the functional response(s) of a particular cell type to a particular functional factor or combination of functional factors may be empirically tested and determined e.g., by culturing the cell type in the presence of the functional factor or combination of functional factors or by contacting the cell with the functional factor or combination of functional factors in vivo, and observing the cells.

### Cells and Cellular Samples

Cells encapsulated in a cellular hydrogel of the instant disclosure include cells produced in a laboratory environment as well as those collected from a living organism. Cells produced in a laboratory environment include but are not limited to cultured cells as well as those cells produced from defined cell culture lines as well as primary cultures. Cells collected from a living organism include but are not limited to those cells collected from a subject or human patient including cells isolated from one or more biological samples collected from the subject or human patient. Collected cells may be used directly, e.g., in the case of a cell therapy where cells are removed from a subject, encapsulated in a hydrogel as described herein and introduced into a subject (i.e., the same or a different subject) without culture and/or expansion. In other instances, collected cells may be used indirectly, including in the case of a cell therapy where cells are removed from a subject, cultured (e.g., culture expanded) and encapsulated in a hydrogel as described herein then introduced into a subject (i.e., the same or a different subject).

In some instances, cells of a cellular sample include stem cells e.g., hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, induced pluripotent stem cells; and the like.

Suitable human embryonic stem (ES) cells include, but are not limited to, any of a variety of available human ES lines, e.g., BG01(hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIH) code, followed in parentheses by the provider code. See, e.g., U.S. Pat. No. 6,875,607.

Suitable human ES cell lines can be positive for one, two, three, four, five, six, or all seven of the following markers: stage-specific embryonic antigen-3 (SSEA-3); SSEA-4; TRA 1-60; TRA 1-81; Oct-4; GCTM-2; and alkaline phosphatase.

Stem cells of the instant disclosure generally include induced pluripotent stem cells. An induced pluripotent stem (iPS) cells is a pluripotent stem cell induced from a somatic cell, e.g., a differentiated somatic cell. iPS cells are capable of self-renewal and differentiation into cell fate-committed stem cells, including neural stem cells, as well as various types of mature cells.

Stem cells of the instant disclosure may further include neural stem cells (NSCs). NSCs are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In some instances, cells of a cellular sample include differentiated cells, including terminally differentiated cells, including those that can be cultured in vitro and used in a therapeutic regimen, where such cells include, but are not limited to, keratinocytes, adipocytes, cardiomyocytes, neurons, osteoblasts, pancreatic islet cells, retinal cells, and the like. In many instances, the cell that is selected for encapsulation in a hydrogel of the instant disclosure will depend in part on the nature of the disorder or condition to be treated.

In some instances, cells of a cellular sample that may be encapsulated in a cellularized hydrogel of the instant disclosure include neurons including but not limited to e.g., sensory neurons, motor neurons, interneurons, unipolar neurons, bipolar neurons, multipolar neurons, anaxonic neurons, Basket cells, Betz cells, Lugaro cells, Medium spiny neurons, Purkinje cells, Pyramidal cells, Renshaw cells, Unipolar brush cells, Granule cells, Anterior horn cells, Spindle cells, Cholinergic neurons, GABAergic neurons, Glutamatergic neurons, Dopaminergic neurons (e.g., midbrain dopaminergic (mDA) neurons), Serotonergic neurons, etc.

In some instances, cells of a cellular sample that may be encapsulated in a cellularized hydrogel of the instant disclosure include dopaminergic progenitors and immature dopaminergic neurons. Such dopaminergic progenitors and immature dopaminergic neurons may include NPCs or other pluripotent cells that have been lineage restricted or partially matured to a dopaminergic fate. In certain instances, dopaminergic progenitors and immature dopaminergic neurons may include but are not limited to such cells described in e.g., Pignatelli et al. Pflugers Arch. (2009) 457(4):899-915; Chung et al. J Neurochem. (2012) 122(2):244-5; Morgan et al. Neuroreport. (2009) 26; 20(13):1225-9; Falkenburger & Schulz. J Neural Transm Suppl. (2006) (70):261-8; Karlsson et al. Cell Transplant. (2005)14(5):301-9; Gao et al. J Neural Transm (Vienna). (1999) 106(2):111-22; Somaa et al. Exp Neurol. (2015) 267:30-41; Doi et al. Stem Cell Reports. (2014) 2(3):337-50; Chung et al. Proc Natl Acad Sci USA. (2011)108(23):9703-8; Morizane et al. J Neurosci Res. (2010) 88(16):3467-78; Akiba et al. J Neurosci Res. (2009) 87(10):2211-21, the disclosures of which are incorporated by reference herein in their entirety. In some instances, the presence of one or more markers, phenotypes, functions or behaviors of dopaminergic progenitors and immature dopaminergic neurons, e.g., as described in the aforementioned references, may be utilized in identifying such cells. In some instances, the absence or one or more markers, phenotypes, functions or behaviors of mature dopaminergic neurons and/or a pluripotent progenitor cell may be used in identifying a dopaminergic progenitor, immature dopaminergic neuron or a population thereof.

In certain embodiments, a cell of cellular sample useful in a cellularized hydrogel of the instant disclosure includes those cells responsive to a particular dispersion factor as described herein. Cells that are responsive to particular dispersion factors are known in the art and also readily ascertainable, e.g., by culturing the cells in the presence of a point-source or gradient of the dispersion factor and observing whether the cells are induced to disperse.

In some instances, the cells of a cell of cellular sample of a cellularized hydrogel of the instant disclosure may exclude cardiac progenitor cells, i.e., the cells of a cellularized hydrogel, in some instances, are not cardiac progenitor cells.

### Kits

Also provided are compositions and kits for use in the subject methods. The subject compositions and kits include any combination of components for performing the subject methods. In some embodiments, a composition can include, but is not limited to and does not require, the following: one or more backbone polymers, one or more linking polymers, one or more functional factors, one or more cellular samples, and any combination thereof.

Components of a subject hydrogel may be present in separate containers. In some instances, two or more components may be combined into a single container. Where two or more components are combined in a single container the components combined may include those components, or a selection thereof, that do not have compatible reactive groups. In some instances, e.g., where two components with compatible components are combined in a single container of a kit the container or the kit as a whole may be kept in conditions that are insufficient for linking of the compatible functional groups including e.g., where the kit or components thereof are kept or stored at a temperature impermissible for linking of the compatible functional groups, where the components of the kit are keep lyophilized or in a buffer impermissible for linking of the compatible functional groups, etc.

Components of a kit or a kit as a whole may be configured for laboratory use where laboratory use may or may not require that the kit components be essentially sterile. In some instances, a kit may be configured for the preparation of an in vitro tissue model cellularized hydrogel where such kits may include but do not necessarily require e.g., instructions for preparing the in vitro tissue model cellularized hydrogel, a mold or other container for preparing the in vitro tissue model cellularized hydrogel, other in vitro tissue culture materials and/or reagents, and the like.

Components of a kit or a kit as a whole may be configured for clinical use where clinical use may require that the kit components be essentially sterile. In some instances, components of the kit may be configured to allow mixing of the components under sterile conditions including e.g., where two or more components may be mixed without exposing the components to environmental air. Kits prepared for clinical use may further include one or more delivery devices including but not limited to e.g., a syringe, a needle, compressible tube, etc. In some instances, one or more components of the kit may come pre-loaded in the delivery device (e.g., the linking agent may be provided in the delivery device). In certain instances, one or more components of the kit may come pre-prepared, including partially pre-prepared, in a single container or in a delivery device

Configuration of kits and components thereof for therapeutic purposes may, in some instances, include the preparation of components for use in humans including e.g., where one or more components of the kit are prepared under xeno-free conditions.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Neural Progenitor Differentiation and Transplantation within 3D Hyaluronic Acid (HA) Biomimetic Extracellular Matrix (ECM)

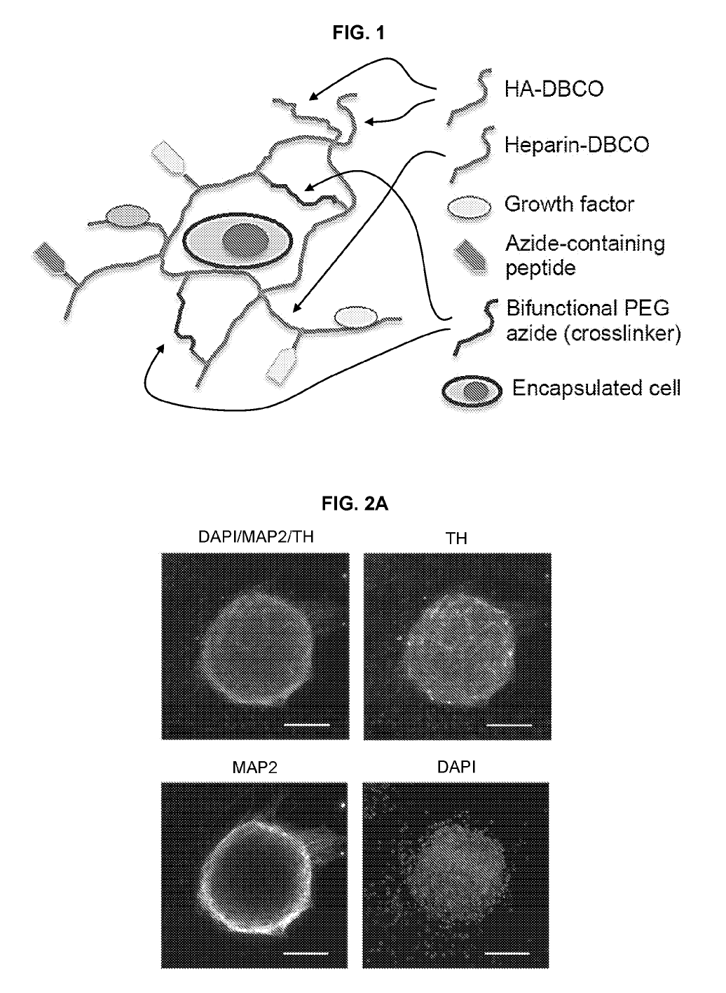

A HA-based hydrogel as described herein was designed as a functionalized biomimetic of the in vivo ECM for in vitro directed differentiation of human pluripotent stem cells (hPSCs) and as a vehicle for carrying cells for therapeutic transplantation into an animal host. As outlined in FIG. 1, the hydrogel comprises HA and heparin functionalized with the requisite peptides and/or proteins that confer biological activity. For instance, the gel can be functionalized with peptides containing an RGD motif that triggers neural cell adhesion, and ephrin-B1, which when presented in a multivalent fashion through HA conjugation, enhances neuronal differentiation. These components were cross-linked to generate a hydrogel of stiffness (i.e. elastic modulus) that is optimized for neuronal differentiation.

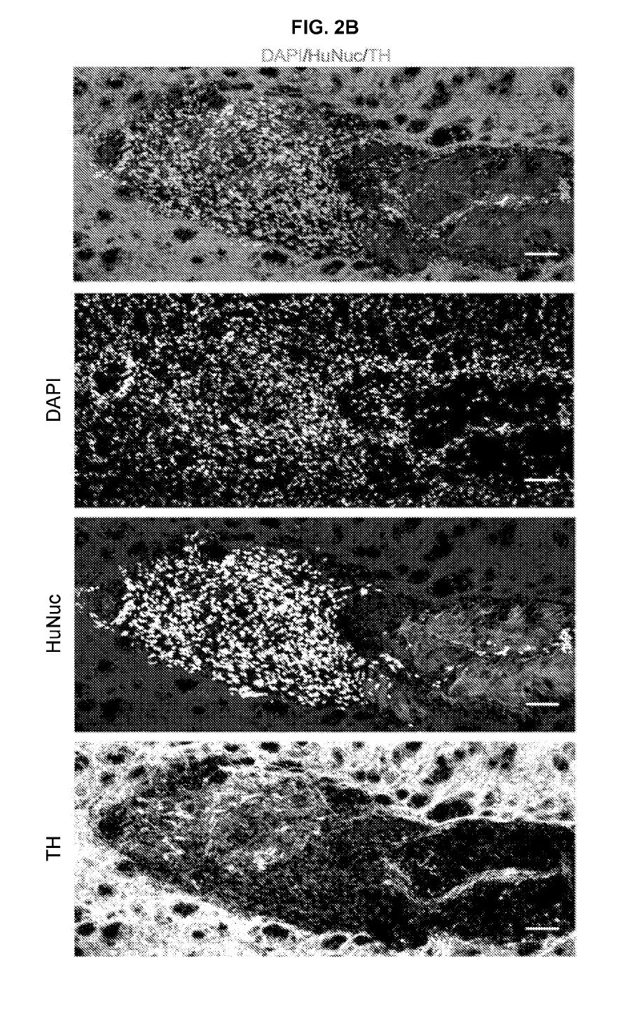

The hydrogel was employed to enhance neuronal lineage restriction and cell viability after transplantation. One method suited to the transplantation of mature neuronal cells, e.g. as a cell therapy for Parkinson's Disease, is described herein. In this example, neural progenitor cells (NPCs) obtained from hPSCs via established protocols (e.g., as described in Chambers et al. (2009) Nat Biotech 27(3):275-280 and Vazin et al. (2008) Stem Cells 26(6):1517-1525; the disclosures of which are incorporated herein by reference in their entirety) were encapsulated within the hydrogels and allowed to differentiate into mature Microtubule Associated Protein-2 (MAP2+) and Tyrosine Hydroxylase-expressing (TH+) dopaminergic (DA) neurons (FIG. 2A) for a period of ˜3 weeks. Then, the cell-laden gel constructs were injected as a plug into the striatum of the host.

In this method, directed neuronal differentiation is enhanced to increase the yield of the desired neuronal phenotype. In addition, the characteristic neural processes formed within the 3D scaffold (FIG. 2A) are retained and neuronal cell function is maintained to a high degree post-transplantation. Hydrogel encapsulation also increases cell survival post-transplantation, as was demonstrated in a rat model (FIG. 2B) using a hyaluronic acid biomaterial.

FIG. 1:

Schematic of click-cross-linked HA ECM platform.

FIG. 2A-2B:

A) NPC differentiation into mature MAP2+/TH+ DA neurons within 3D HA ECMs. B) Survival of Human Nuclear Antigen (HuNuc)+/TH+ DA neurons encapsulated in 3D HA hydrogels 8 weeks after transplantation into the striatum of rats. (Scale bar—100 mm).

### Example 2: 3D Hydrogels for Improved Post-Transplantation Survival of hPSC Derived Midbrain Dopaminergic (mDA) Neurons

### Materials and Methods

The following materials and methods generally apply to the results presented in Example 2 except where noted otherwise. HA gel synthesis HA hydrogels were prepared using the Strain Promoted Azide Alkyne Cycloaddition (SPAAC) reaction to effect rapid crosslinking and gelation. First, Hyaluronic Acid (HA) was functionalized with dibenzocyclooctyne (DBCO) by reacting Sodium Hyaluronate (average molecular weight 75 kDa, Lifecore Biomedical) with DBCO-amine (Sigma-Aldrich). Briefly, 500 mg HA was dissolved at 1 mg/mL in 2-(N-Morpholino)ethanesulfonic acid (MES) buffer (50 mM, pH 4.0) and the carboxylic acid groups were activated by an equimolar amount of N-(3-Dimethylaminopropyl)-N′-ethylcarbodiimide (EDC) and N-hydroxysuccinide (NHS) for 1 h. DBCO-amine was dissolved in dimethylsulfoxide (DMSO) and 0.6 equivalents were added dropwise with stirring. After 48 h reaction at room temperature, the reaction mixture was concentrated using a spin filter with a 10 kDa cutoff and HA-DBCO was precipitated out by adding a fivefold excess of cold acetone. The precipitate was pelleted by centrifugation, washed once with cold acetone, and then dissolved in ultrapure water and lyophilized. Heparin-DBCO was similarly synthesized starting with porcine heparin (Sigma-Aldrich). 1H NMR was used to estimate the extent of functionalization of HA and Heparin with DBCO groups (approximately 10%).

For making hydrogels, HA-DBCO was dissolved in phosphate-buffered saline (PBS) and functionalized with an azide-containing RGD peptide (K(az)GSGRGDSP (SEQ ID NO:2), Genscript; where K(az) stands for azidolysine) to a final concentration of 0.5 mM. 0.07% (w/w of HA-DBCO) of heparin-DBCO dissolved in PBS was added, and finally the polymers were cross-linked using homobifunctional PEG-azide (average molecular weight 1 kDa, Creative Pegworks). The amount of HA-DBCO (w/w %) was varied to obtain gels of different stiffnesses. For encapsulating cells within the hydrogels, cell pellets were resuspended in HA-RGD-Heparin gels before cross-linking with PEG-azide.

### Dopaminergic Differentiation

hPSCs were differentiated to dopaminergic progenitors and immature dopaminergic neurons on pNIPAAM-PEG 3D gels (Mebiol, Cosmobio, USA) using a protocol adapted from previously established differentiation techniques described in e.g., Kriks, S. et al. (2011) Nature 480:547-551 and Kirkeby, A. et al. (2012) Cell Rep. 1:703-714; the disclosures of which are incorporated herein by reference in their entirety. 5 days after singe cell passage and maintenance in supplemented E8 with 10 μM ROCK inhibitor (Y27632, Fischer), differentiation was initiated with Dual-SMAD inhibition using 100 nM LDN193189 (Stemgent, San Diego, Calif.) and 10 μM SB431542 (Selleckchem, Carlsbad, Calif.). Medium conditions were maintained throughout differentiation. After 15 days in pNIPAAM-PEG, cells were harvested with cold PBS and pipet mixed to small ˜100 μm clusters, which were then seeded onto a 0.01% poly-L-ornithine (Sigma, St. Louis, Mo.)/20 μg/ml laminin (Invitrogen, Grand Island, N.Y.)) coated plate for 2D culture, or encapsulated in HA gels for 3D culture. N2 (Life technologies, Grand Island, N.Y.), B27 (Life technologies, Grand Island, N.Y.), Glutamax (Invitrogen, Grand Island, N.Y.), 100 ng/ml FGF8 (Peprotech, Rocky Hill, N.J.), 3 μM CHIR99021 (Stemgent, San Diego, Calif.), 20 ng/ml BDNF (Peprotech, Rocky Hill, N.J.), 20 ng/ml GDNF (Peprotech, Rocky Hill, N.J.), 2 μM Purmorphamine (Stemgent, San Diego, Calif.), 0.5 mM DibutyrylcAMP (Santa Cruz Biotechnologies, Dallas, Tex.), 10 μM DAPT (Selleckchem, Carlsbad, Calif.), 1 ng/ml TGFβ3 (R&D Systems, Minneapolis, Minn.) and 0.2 mM L-Ascorbic Acid (Sigma-Aldrich, St Louis, Mo.) were used in medium formulations as needed.

### Quantitative Immunocytochemistry

On D15 of differentiation, mDA progenitors and immature dopaminergic neurons were harvested from the pNIPAAM-PEG platform by liquefying the gel with cold PBS. Cells were then seeded on 0.01% poly-L-ornithine (Sigma, St. Louis, Mo.)/20 μg/ml laminin (Invitrogen, Grand Island, N.Y.)) coated plate. The next day, cells were fixed with 4% paraformaldehyde. At days 25 and 40 of mDA differentiation, cells on 2D surfaces (laminin-coated plates) or 3D HA gels were fixed with 4% paraformaldehyde. Following three washes with PBS, cells were blocked for 1 h at RT on a rocker with primary blocking buffer (2% BSA, 5% donkey serum, 0.3% Triton ×100 in PBS). Primary antibodies diluted in primary blocking buffer were incubated with the cells, overnight for 2D cells and for 48h for 3D gels, on a rocker at 4° C. Next, cells were rinsed once with 0.2% Triton in PBS and washed three times with 0.1% Triton in PBS, followed by a 2h incubation with appropriate secondary antibodies diluted in 2% BSA in PBS. DAPI was added 30 min before the end of secondary antibody incubation period. Cells were subsequently washed three times with PBS and imaged on a Zeiss fluorescent microscope for 2D cultures. The various primary and secondary antibodies used and their respective dilutions are presented herein in Table 1. For the transcription factors FOXA2, LMX1A, MSX1, PAX6, OCT4 and NANOG, the number of cells labeled positive was counted in Cell Profiler and expressed as a percentage of total DAPI labeled cells in the image. Percentage of cells positive for neuronal markers Tuj1 and TH were manually counted using the cell counter feature in ImageJ.

qPCR

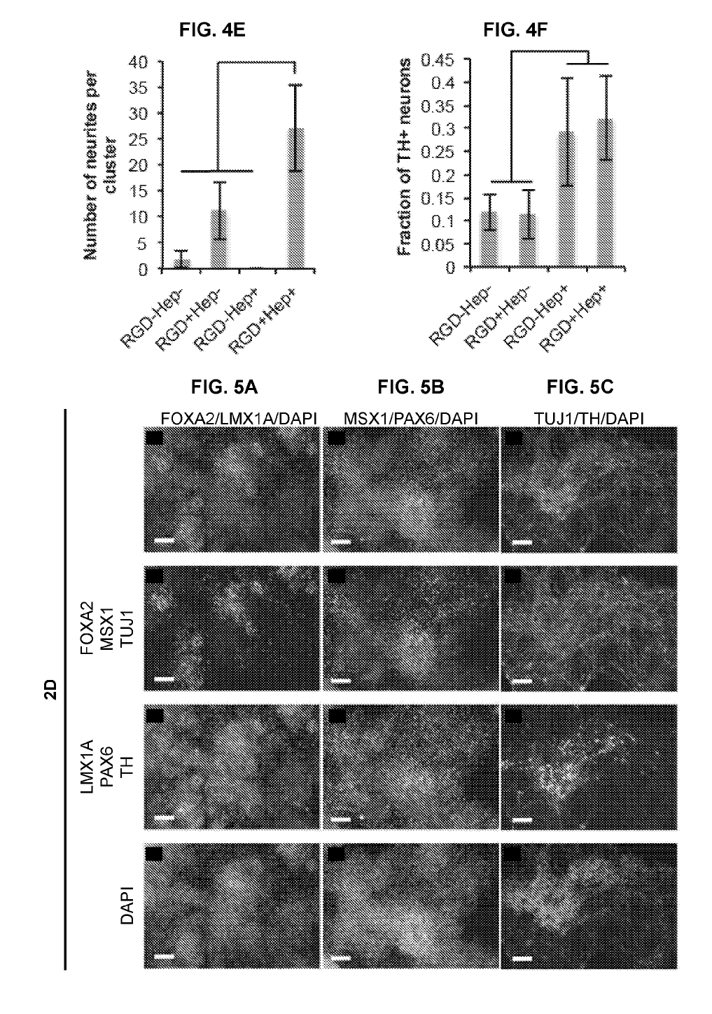

At days 25 (for 2D and 3D HA cultures) and 40 (for 3D HA cultures) of differentiation, cells were harvested and mRNA extracted using Qiagen RNA extraction kit (Qiagen) according to the manufacturer's instructions. mRNA was reverse-transcribed using iScript reverse transcriptase (Biorad), and quantified on an iQ5 RT-PCR detection system (Biorad). Data was normalized to GAPDH expression and analyzed using the 2^{ΔΔCt} method. The primers used for qPCR are presented in Table 2 and representative data is provided in FIG. 5G-5H.

### In Vitro Injection Tests

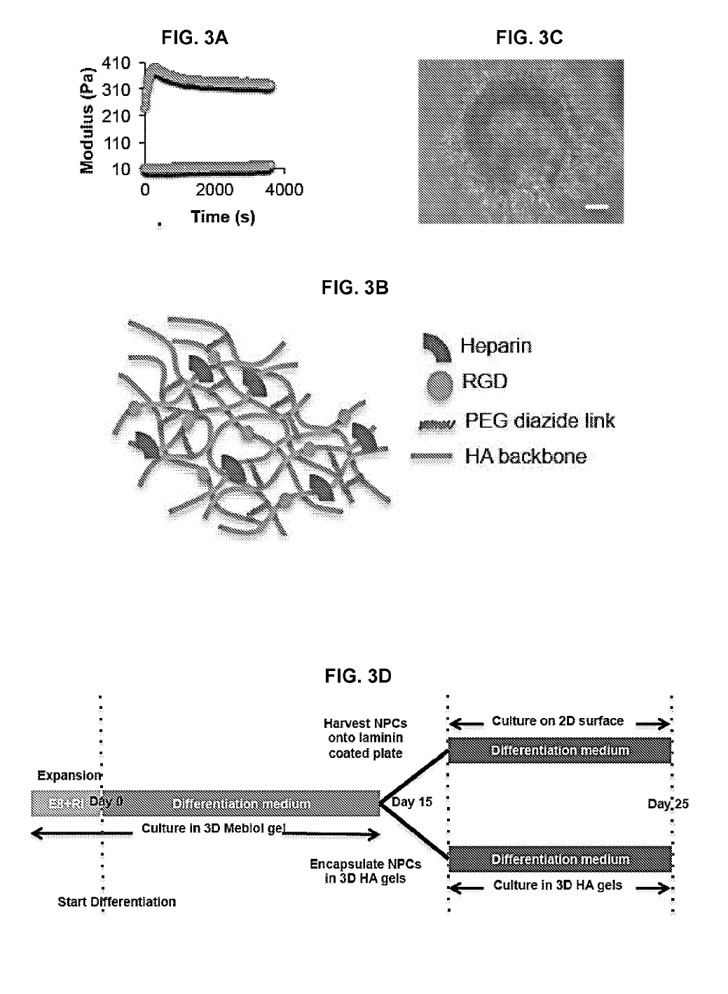

mDA neuron precursors were generated in 3D PEGNIPAM (herein also referred to as Mebiol) and then transferred onto 2D or 3D platforms, as depicted in FIG. 3D. Injection tests were performed at D25 of differentiation. For both the 2D and 3D samples, cells were analyzed at three different stages: 1) before harvest 2) after harvest and reseeding 3) after harvest, post-injection and reseeding. Cells were harvested from the 2D platform using 0.5 mM EDTA and gentle mechanical scraping, and either reseeded directly or post-injection through a 26 gauge needle on a 10 μl Model 701 RN glass syringe (Hamilton) onto a laminin coated surface. Cells from 3D were collected by simply pipetting up the 3D gel, with the cells encapsulated within, and either reseeding directly or post injection through the same 26 gauge needle onto a plate. Cells remained encapsulated during harvest, injection and reseeding. For a separate subset of samples, cells were harvested from 2D, encapsulated in 3D HA gels, and either reseeded directly or post injection onto a plate. After reseeding, all samples were fed with 1:50 B27 supplemented Neurobasal medium. 24h later, LDH assay was used to measure the total cell counts for each sample following the manufacturer's protocol (Promega). The percentage of dead cells (x) was estimated from the LDH activity in the supernatant, and the percentage of live cells (y) was estimated from the LDH activity post-lysis of any remaining cells. The percentage of cells lost during the harvesting/injection/reseeding processes was calculated as 100−x−y.

### In Vivo Transplantations

For 2D bolus injections, D25 mDA neurons were harvested from 2D laminin coated surfaces, and dissociated to small ˜50-100 μm clusters using 0.5 mM EDTA and pipetting. For injections of HA encapsulated cells, 3D gels were first loaded into the backend of the syringe using a positive displacement pipet before injections. For all injections, a 26 gauge needle on a 10 μl Model 701 RN glass syringe (Hamilton) was used. 100000 cells were implanted into the striatum of isoflurane anesthetized 150-200 g adult female Fischer 344 rats (at stereotaxic coordinates AP: +1.0, ML: −2.5, DV −5.0). 10 mg/kg Cyclosporine was injected intraperitoneally daily starting 24h before surgeries and until the animals with euthanized. 6 weeks after cell implantations, animals were intracardially perfused with 4% PFA. Brains were harvested and incubated in 4% PFA overnight, and transferred into a 30 (w/v) % sucrose solution the following day.

4-5 days later, after the brains were sufficiently dehydrated and had sunk to the bottom of the containers, they were sliced into 40 μm sections using a microtome. Primary antibodies diluted in primary blocking buffer (5% donkey serum, 2% BSA, 0.1% Triton ×100) were incubated with the brain sections for 72h with gentle rocking at 4° C. Following incubation, brain sections were rinsed once with 0.2% Triton in PBS and washed three times with 0.1% Triton in PBS, followed by a 4h incubation with appropriate secondary antibodies diluted in 2% BSA in PBS. DAPI was added 30 min before the end of secondary antibody incubation period. Brain sections were subsequently washed three times with PBS and mounted on coverslips. A Zeiss Axioscan Z1 automated slide scanner was used to image the brain sections, and Zen 2.0 software was used to analyze the images. For high resolution imaging, a Zeiss AxioObserver fluorescent microscope was used.

Percentage of cell survival was quantified using the cell counter feature on ImageJ, as described in Kirkeby, A. et al. (2012) Cell Rep. 1:703-714; the disclosure of which is incorporated herein by reference in its entirety, using a method based on Abercrombie's technique (Abercrombie, M. (1946) Anat. Rec. 94:239-247; the disclosure of which is incorporated herein by reference in its entirety). All cells positive for HNA and TH were counted from zoomed in pictures originally acquired at 5× magnification on the Zeiss Axioscan slider scanner, of every 5^{th} brain section spanning the injection site (˜8 sections across ˜50 total sections). The total number of HNA positive and TH positive cells were then extrapolated from these counts. Furthermore, all HNA positive cells were counted from three representative sections for each rat brain, imaged at 20× magnification on the Zeiss AxiObserver. Cells double positive for TH/HNA and FOXA2/HNA were then identified for these pictures and counted. To avoid double counting, any cells out of focus were disregarded.

### Results

### HA Gels for mDA Development

By controlling the following factors i) HA polymer weight fraction (w/v %), ii) fraction of heparin (w/w of HA) and iii) fraction of cross linker PEG-diazide (w/w of HA), the stiffness of the resulting gel was modulated as desired. Specifically, for ˜10% DBCO functionalized HA, at 3.5 w/v % HA-DBCO, 0.07% wt Heparin-DBCO/wt HA-DBCO and 0.07% wt PEG-diazide/wt HA-DBCO gels of stiffness ˜350 Pa were obtained (FIG. 3A).

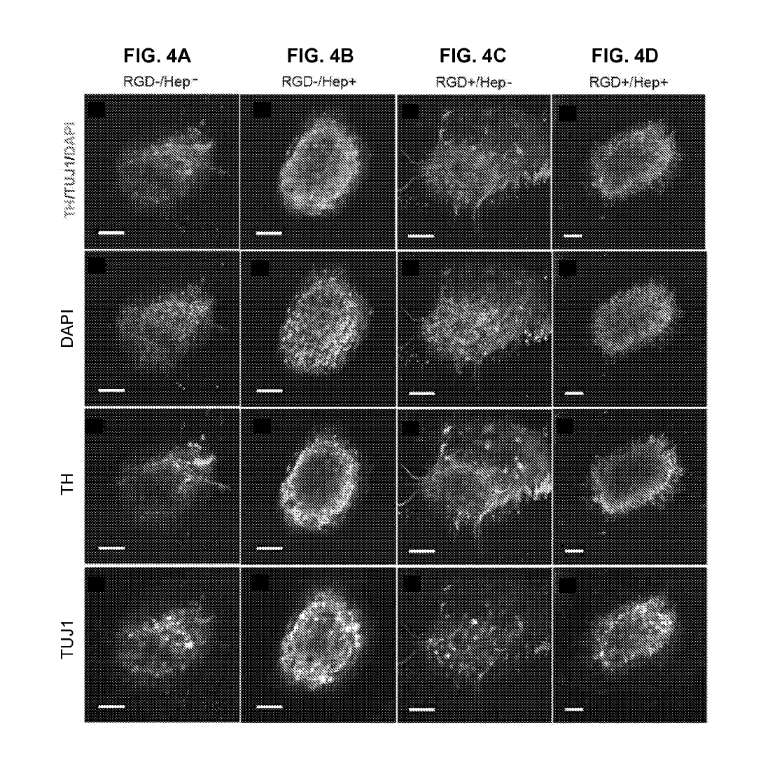

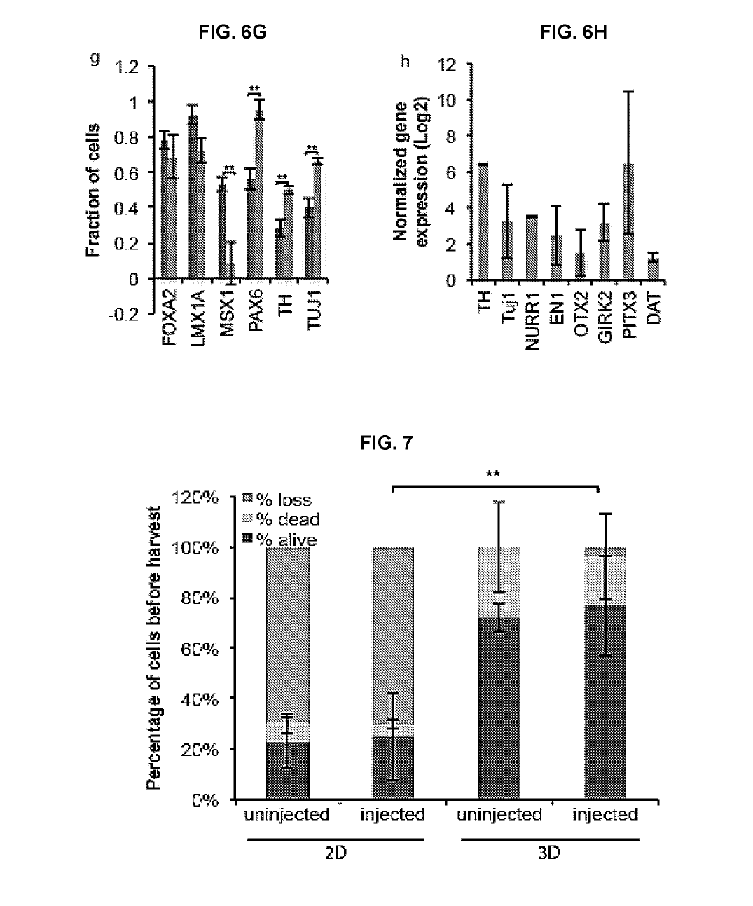

Next, the effect of RGD and heparin functionalization of HA gels on mDA neuronal development was investigated. The design of the gel is schematically depicted in FIG. 3B. hESCs were differentiated for 15 days into mDA neuronal progenitors and immature neurons in 3D Mebiol gels, and encapsulated within HA gels and cultured till D25 (FIG. 3C). The differentiation protocol is schematically depicted in FIG. 3D. Four different types of HA gels were used: i) non-functionalized HA ii) RGD+ HA iii) Heparin+ HA iv) RGD+ Heparin+ HA. At D25, cells were characterized using immunocytochemistry (FIG. 4A-4F). It was found that HA gels functionalized with RGD showed a higher number of neurites per cluster compared to gels without RGD (FIG. 4E). Furthermore, gels dual-functionalized with both RGD and heparin showed significantly higher number of neurites compared to all other conditions. Therefore, while the level of neurite outgrowth is increased in HA gels functionalized with RGD alone, it is significantly higher in gels with both RGD and heparin. Additionally, gels with heparin, with or without the presence of RGD, significantly increased the fraction of TH+ cells (FIG. 4F). The neurotrophic environment provided by heparin functionalization, by binding and retaining factors such as GDNF, can therefore improve dopaminergic differentiation. mRNA levels of other markers of interest, such as PAX6 for neuronal commitment (see e.g., Chambers et al. (2009) Nat. Biotechnol. 27:275-280; the disclosure of which is incorporated herein by reference in its entirety), FOXA2 and LMX1A for floorplate-derived midbrain regional specification (see e.g., Kriks et al., (2011) Nature 480:547-551; the disclosure of which is incorporated herein by reference in its entirety), and NURR1 and DAT for dopaminergic maturation (see e.g., Hegarty et al. (2013) Dev. Biol. 379:123-138; the disclosure of which is incorporated herein by reference in its entirety) did not show significant differences between the different conditions, although higher expression of some markers was noted in HA gels dual functionalized with heparin and RGD (FIG. 9). Therefore, for all subsequent work in this study with HA gels, RGD and heparin functionalization were used.

### Characterization of mDA Neuronal Maturation on 2D Surfaces Versus in 3D HA Gels

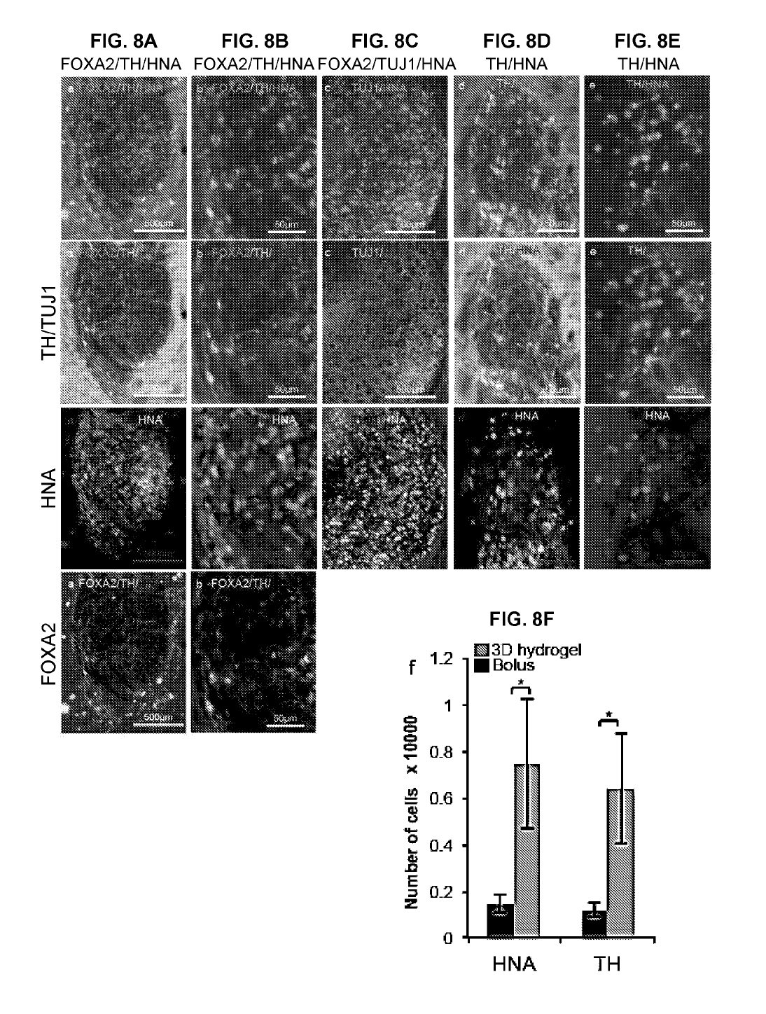

mDA neuron progenitors were generated on Mebiol gels for 15 days (FIG. 10) (e.g., as previously described Lei & Schaffer (2013) Proc. Natl. Acad. Sci. U.S.A. 110:E5039-E5048; the disclosure of which is incorporated herein by reference in its entirety), and then transferred onto 2D Poly-L-Ornithine/laminin coated surfaces or encapsulated in 3D HA gels as depicted in FIG. 3D. Immunocytochemistry and qPCR at D25 (FIG. 5A-5H) showed expression of relevant markers of interest in both platforms. Markers indicative of a floorplate derived midbrain fate, FOXA2 and LMX1A, were expressed at high levels in both the 3D platform and 2D platforms (FIG. 5G). FOXA2 expression, however, was significantly higher in the 3D platform. However, tyrosine hydroxylase, TH, the rate-limiting enzyme for dopamine production in mDA neurons, was expressed at equivalent levels in both platforms. qPCR analysis further confirmed that there was no significant difference in expression of several markers of interest, LMX1A, PAX6, TH, TUJ1 between the 2D and 3D platforms (FIG. 5H). Thus, with the exception of FOXA2 expression, the mDA neuronal fate is equivalently maintained in both the 2D and 3D HA platforms after transfer from the 3D pNIPAAM-PEG gels at D15.

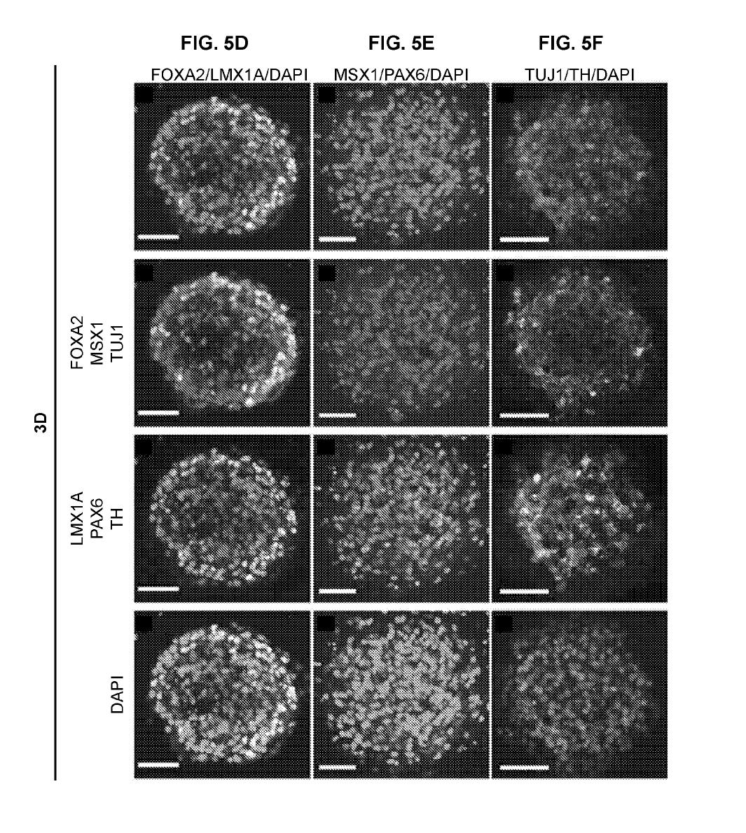

The long-term maintenance of mDA phenotype was assessed using immunocytochemistry after culture in the 3D or 2D platforms until Day 40 of differentiation (FIG. 6A-6G). High levels (˜70-90%) FOXA2/LMX1A expression was observed in both platforms, demonstrating continued maintenance of the floorplate-derived midbrain fate. Interestingly, both the dopaminergic marker TH and the pan neuronal marker TUJ1 were expressed at a higher level in 3D compared to 2D at this stage. Additionally, qPCR analysis at D40 of cells differentiated in 3D HA gels confirmed the expression of mature mDA neuronal markers DAT, GIRK2 and PITX3 (FIG. 6H). Thus, the RGD and heparin functionalized 3D HA gels provide a neurogenic environment amenable for the long-term development and maturation of mDA neurons.

### In Vitro Injection Tests

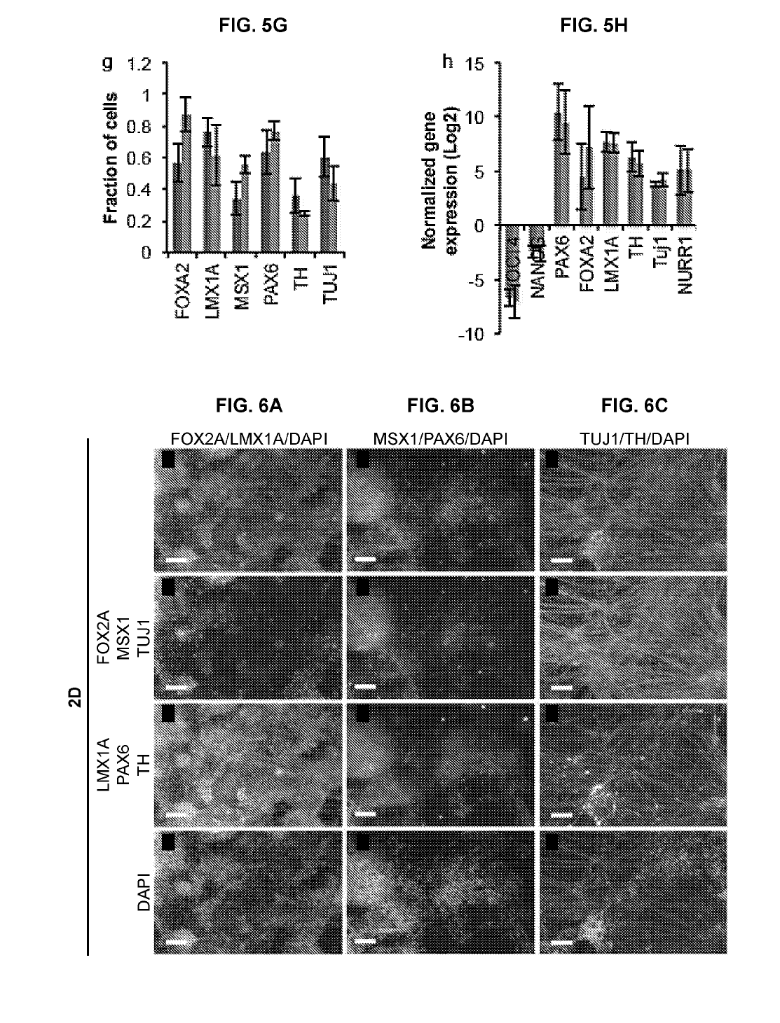

To recapitulate in vitro the differing in vivo effects of 2D vs. 3D cell delivery, the viability of mDA neurons harvested from 2D surfaces and injected through a needle was measured and compared to the viability of cells going through the same process while encapsulated in a HA gel. As uninjected controls, cells from both platforms were collected and reseeded without injection. Results indicate that 70% of the cells are lost during the process of harvesting from 2D, compared to less than 3% when collecting from 3D gels (FIG. 7). Losses at this stage are likely due to i) cell loss during centrifugation and transfers and ii) cell death due to adverse mechanical, chemical or hypoxic stresses. Encapsulation within 3D gels eliminates all of these issues.

Interestingly for both the 2D and 3D systems, injection resulted in no significant reduction in cell viability. About 70% of the cells were viable in both systems (“dead” as a fraction of “alive”), after cells were harvested and reseeded with or without injection. Again, cell losses at this stage are therefore attributed to losses arising from transfers rather than stress from injection.

### In Vivo Survival

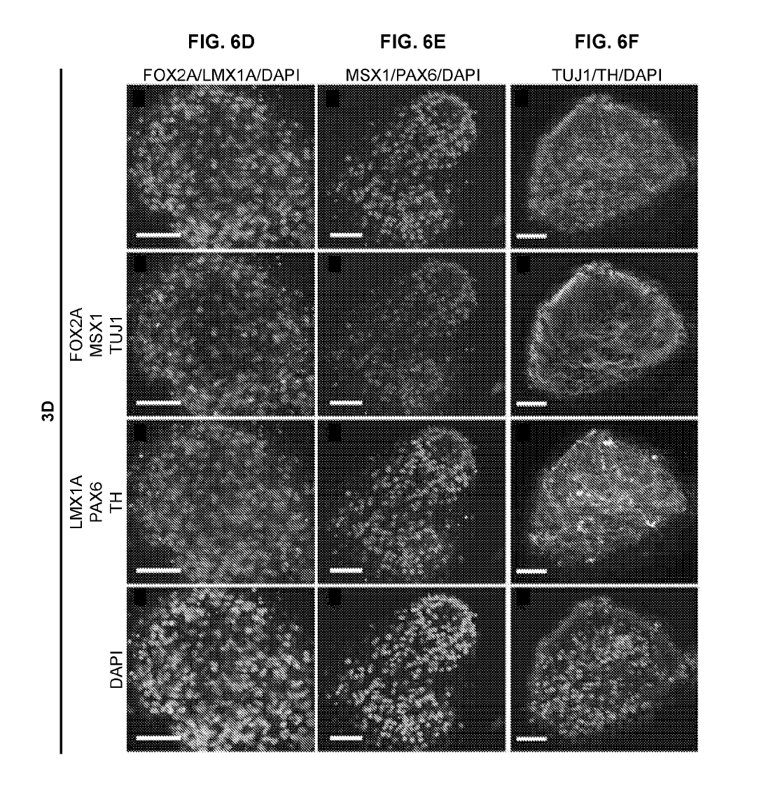

Immunohistochemical analysis of brain sections 4.5 months post-transplantation indicated robust numbers of HNA positive cells with a high fraction co-expressing TH and FOXA2, demonstrating the continued maintenance of a floorplate-derived midbrain dopaminergic fate (FIG. 8A-8F). On average, 1444±386 HNA positive cells (corresponding to ˜1% of transplanted cells) survived when transplanted as a bolus injection post-harvest from a 2D laminin-coated surface. In contrast, 7487±2791 HNA positive cells (corresponding to ˜10% of transplanted cells) survived when transplanted in 3D HA hydrogels. Out of the surviving HNA positive cells, on average 1195±319 cells (˜80% of surviving cells, and 1.6% of transplanted cells) were TH positive in the 2D bolus implants. In contrast, 6401.7±2385 TH+ neurons (corresponding to ˜85% of surviving cells, and 8% of transplanted cells) survived in the 3D HA hydrogels. The level of TH+ neuronal survival is therefore significantly higher in the 3D transplantations compared to current standards in the field represented by the 2D bolus injections. Additionally, although both the 2D and 3D grafts showed a high fraction of TH positive cells, higher levels of neurite outgrowth was seen for the 3D implants (FIG. 8D, FIG. 8E). Differentiation in the 3D pNIPAAM-PEG platform accelerates the maturation of mDA neurons compared to neurons generated on 2D surfaces. Consequently, mDA progenitors generated in the 3D pNIPAAM-PEG platform and matured on 2D or 3D platforms until D25 are at a higher level of maturity compared to cells standardly differentiated solely on 2D platforms. The results presented here show that encapsulation within the functionalized 3D HA platform can successfully rescue the survival of mature mDA neurons.

### FIG. 3A-3D:

Characterization of HA hydrogels and differentiation of mDA neurons. a) Storage and Loss modulus of RGD and heparin functionalized HA hydrogels constructed with 3.5% w/v HA b) Pictorial depiction of RGD and heparin functionalized HA hydrogel cross-linked with PEG diazide cross linkers. c) mDA neuron cluster growing within HA hydrogel. Scale bar=100 μm d) Schematic depicting generation of mDA neurons from hESCs in 3D vs 2D.

### FIG. 4A-4F:

Effect of incorporating RGD and Heparin on mDA development and 3D neuronal cluster morphology. Confocal images showing expression of TH and TUJ1 in mDA neuronal clusters cultured within HA hydrogels a) without RGD and Heparin b) functionalized with Heparin c) functionalized with RGD and d) functionalized with RGD and Heparin. e) Number of extended neurites per mDA neuronal cluster for mDA neurons cultured within various HA hydrogels; Scale bars=100 μm. f) Fraction of cells expressing TH in mDA neurons cultured within various HA hydrogels.

### FIG. 5A-5H:

Characterization of D25 mDA neurons matured in 2D or 3D HA hydrogels. a-f) Representative fluorescence images highlighting significant differences between 2D and 3D cultures; (a,d) FOXA2/LMX1A and (b,e) MSX1/PAX6 and Tuj1/TH. g) Quantitative immunocytochemistry comparing mDA marker expression at Day 25 between 2D (left bars) and 3D (right bars) cultures. Data are presented as mean±s.e.m. for n=3 independent experiments. *=p<0.05 for Student's t test. Nuclei are labeled with DAPI. Scale bars, 100 μm. h) Comparative gene expression analysis at Day 25 between 2D (left bars) and 3D (right bars) generated mDA neurons. Data are presented as mean±standard deviation from triplicates.

### FIG. 6A-6H:

Characterization of D40 mDA neurons matured in 2D or 3D HA hydrogels. a-f) Representative fluorescence images highlighting significant differences between 2D and 3D cultures; (a,d) FOXA2/LMX1A and (b,e) MSX1/PAX6 and Tuj1/TH. g-h) Quantitative immunocytochemistry comparing mDA marker expression at Day 40 between 2D (left bars) and 3D (right bars) cultures. Data are presented as mean±s.e.m. for n=3 independent experiments. *=p<0.05 for Student's t test. Nuclei are labeled with DAPI. Scale bars, 100 μm. h) Gene expression analysis at Day 40 for 3D generated mDA neurons. Data are presented as mean±standard deviation from triplicates.

### FIG. 7:

In vitro LDH test as a measure of mDA neuron survival post-harvest and injection. LDH levels were measured at different stages for cells harvested from 2D or 3D platforms and reseeded onto 2D plates or 3D HA hydrogels with or without injection through a 26 gauge needle, and presented as a percentage of total cells prior to harvest. Percentage of live cells (bottom portion of stacked bars), dead cells (middle portion of stacked bars) and lost cells (top portion of stacked bars) were calculated for each condition. Data are presented as mean±s.e.m. for n=3 independent experiments. **=p<0.005 for Student's t test.

### FIG. 8A-8F:

In vivo survival of mDA neurons transplanted with or without encapsulation within 3D HA hydrogels. a) Graft morphology at 4.5 months post-transplantation, showing expression of HNA, TH, and FOXA2. b) Coexpression of TH and FOXA2 in surviving HNA+ cells. c) Coexpression of TUJ1 and HNA. Coexpression of TH and HNA in neurons transplanted d) encapsulated in 3D hydrogels or e) as a bolus injection. f) Quantification of total number of HNA+ and TH+ surviving cells from 8 animals/group for neurons transplanted encapsulated in 3D hydrogels (right bars) or as a bolus injection (left bars). Data are presented as mean±s.e.m. *=p<0.05 for Student's t-test.

### FIG. 9:

Effect of RGD and heparin functionalization of HA platforms on mDA neuronal maturation. Gene expression analysis at Day 25 for mDA neurons matured on different HA platforms. Data are presented as mean±standard deviation from triplicates.

### FIG. 10:

Immunocytochemistry of D15 mDA neurons generated on 3D pNIPAAM-PEG platform. Fluorescence images showing expression of a) FOXA2/LMX1A, b) MSX1/PAX6 and OCT4/NANOG; images are representative of n=3 independent experiments. Scale bars=100 μm.

TABLE 1
Antibodies used in immunocytochemistry.

| Antibodies | Company | Cat. No. | Host | Dilution |
|---|---|---|---|---|
| FoxA2 | Santa Cruz | sc-101060 | Mouse | 1:500 |
| Lmx1A | Millipore | MAB10533 | Rabbit | 1:500 |
| Tuj1 | Invitrogen | 480011 | Mouse | 1:500 |
| TH | Pel-freeze | P40101 | Rabbit | 1:500 |
| OCT4 | Santa Cruz | sc-5279 | Mouse | 1:200 |
| NANOG | Santa Cruz | sc-33759 | Rabbit | 1:200 |
| MSX1 | Hybridoma bank | 4G1-C | Mouse | 1:100 |
| PAX6 | Biolegend | PRB-278P | Rabbit | 1:300 |
| TH | Abcam | ab76442 | Chicken | 1:1000 |
| Alexa 647 Donkey | Jackson | A31571 | Donkey | 1:1000 |
| anti Ms | Immunoresearch | | | |
| Alexa 555 Donkey | Jackson | A31572 | Donkey | 1:1000 |
| anti Rb | Immunoresearch | | | |
| Alexa 488 Donkey | Jackson | 703-545-155 | Donkey | 1:1000 |
| anti Ch | Immunoresearch | | | |

TABLE 2
Primers used in qPCR.

| | | | Amplicon | Tm_{F}; |
|---|---|---|---|---|
| Gene | Forward primer (F) | Reverse primer (R) | size | Tm_{R} |
| OCT4 | CACCATCTGTCGCTTCGAG | AGGGTCTCCGATTTGCAT | 132 | 62.6; |
| | G (SEQ ID NO: 11) | ATCT (SEQ ID NO: 12) | | 60.7 |
| NANOG | AAGGTCCCGGTCAAGAAA | CTTCTGCGTCACACCATT | 237 | 62; 61.9 |
| | CAG (SEQ ID NO: 13) | GC (SEQ ID NO: 14) | | |
| PAX6 | AACGATAACATACCAAGC | GGTCTGCCCGTTCAACAT | 120 | 60; 60.8 |
| | GTGT (SEQ ID NO: 15) | C (SEQ ID NO: 16) | | |
| FOXA2 | GGAGCAGCTACTATGCAG | CGTGTTCATGCCGTTCAT | 83 | 62.3; |
| | AGC (SEQ ID NO: 17) | CC (SEQ ID NO: 18) | | 61.7 |
| OTX2 | CATGCAGAGGTCCTATCCC | AAGCTGGGGACTGATTGA | 200 | 60.8; |
| | AT (SEQ ID NO: 19) | GAT (SEQ ID NO: 20) | | 60.6 |
| PITX3 | CCTACGAGGAGGTGTACC | CCCACGTTGACCGAGTTG | 112 | 62.6; |
| | CC (SEQ ID NO: 21) | A (SEQ ID NO: 22) | | 61.9 |
| DAT | TTTCTCCTGTCCGTCATTG | AGCCCACACCTTTCAGTA | 223 | 62.4; |
| | GC (SEQ ID NO: 23) | TGG (SEQ ID NO: 24) | | 61.8 |
| TH | GGGCTGTGTAAGCAGAAC | AAGGCCCGAATCTCAGGC | 107 | 60.7; 63 |
| | G (SEQ ID NO: 25) | T (SEQ ID NO: 26) | | |
| NURR1 | ACCACTCTTCGGGAGAAT | GGCATTTGGTACAAGCAA | 175 | 60; 61.1 |
| | ACA (SEQ ID NO: 27) | GGT (SEQ ID NO: 28) | | |
| GIRK2 | CACATCAGCCGAGATCGG | GGTAGCGATAGGTCTCCC | 103 | 60.3; |
| | AC (SEQ ID NO: 29) | TCA (SEQ ID NO: 30) | | 61.2 |
| TUJ1 | GGCCAAGGGTCACTACAC | GCAGTCGCAGTTTTCACA | 85 | 62.3; 62 |
| | G (SEQ ID NO: 31) | CTC (SEQ ID NO: 32) | | |
| LMX1A | ACGTCCGAGAACCATCTT | CACCACCGTTTGTCTGAG | 248 | 61.8; 61 |
| | GAC (SEQ ID NO: 33) | C (SEQ ID NO: 34) | | |
| EN1 | GAGCGCAGGGCACCAAAT | AATAACGTGTGCAGTACA | 138 | 62.7; |
| | A (SEQ ID NO: 35) | CCC (SEQ ID NO: 36) | | 60.3 |
| GAPDH | GGAGCGAGATCCCTCCAA | GGCTGTTGTCATACTTCT | 197 | 61.6; |
| | AT (SEQ ID NO: 37) | CATGG (SEQ ID NO: 38) | | 60.9 |

### Example 3: Dispersion Factors for Use in Cell Transplantation

### Results

A major challenge facing efficient cell replacement therapy is ineffective dispersion of cells from the injection site post-transplantation. For example, in Parkinson's Disease, poor treatment outcome and undesirable side effects have been attributed to low levels of integration and localized graft hotspots resulting from ineffective dispersion of transplanted cells. To address this issue, hyaluronic acid (HA) based hydrogels functionalized with appropriate dispersion factors and modified to have physical properties that promote dispersion were developed to effectively transplant hydrogel-encapsulated neurons into the central nervous system.

In the instant example, a 3D biomaterial transplantation platform for increasing integration and dispersion of cells post-transplantation was designed and tested. Generally, the platform includes HA polymers cross-linked with a PEG linker. For added functionality and broad applicability, biochemical cues were covalently added to the HA backbone, cross-linked using the PEG linker, or physically encapsulated within the gel. In certain instances, the biophysical and biochemical properties of the HA hydrogel can be adjusted to tune the system for encapsulation of a variety of different cell types and to improve the effectiveness of in vivo transplantation.

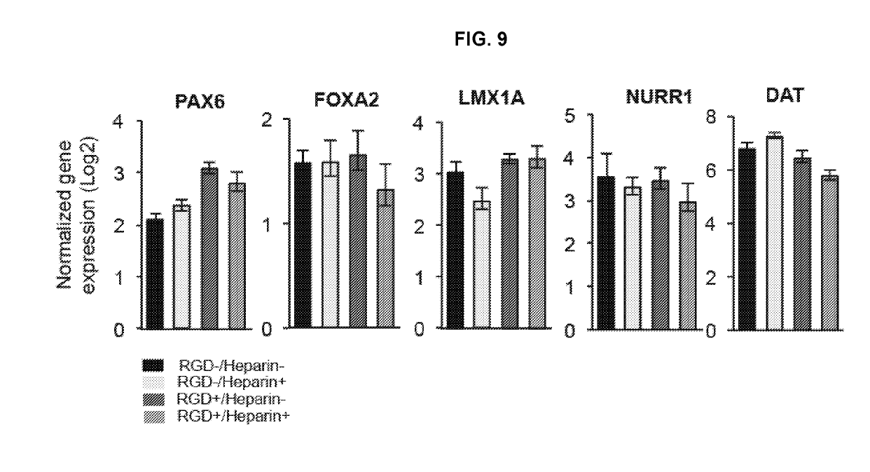

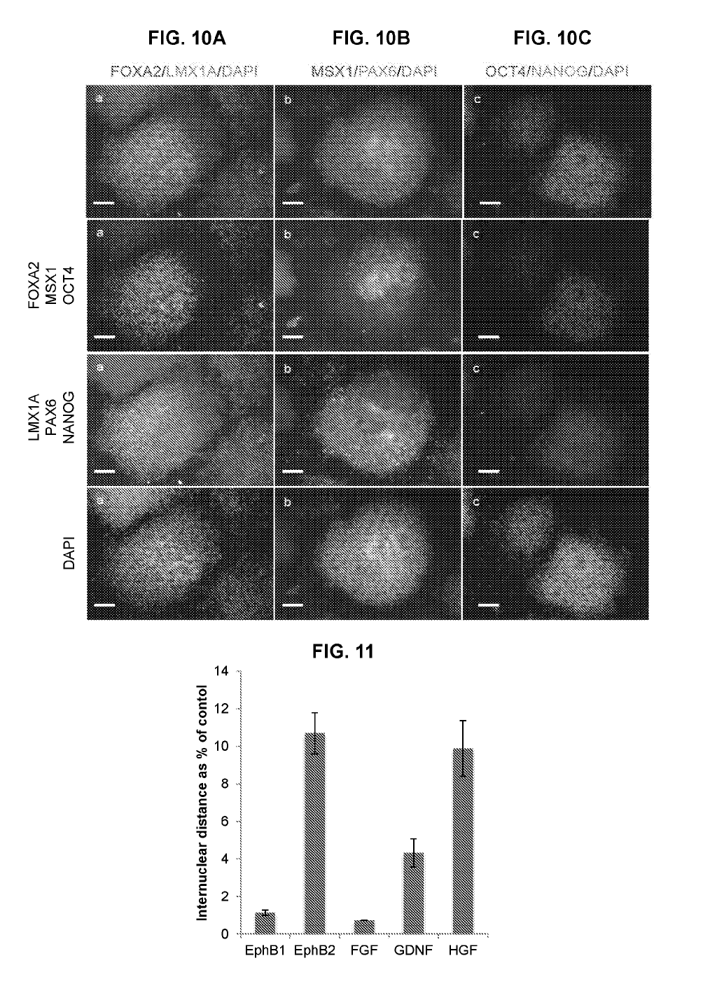

Three factors, Hepatocyte growth factor (HGF), Glial derived neurotrophic factor (GDNF) and EphrinB2, were identified that most effectively dispersed midbrain dopaminergic neurons (FIG. 11). Subsequently, optimal concentrations of the dispersion factors for the tested cell types were identified (FIG. 12). It was observed that while dispersive effects were equivalent between the different concentrations tested after 10 days of dispersion, the middle concentrations of HGF (20 ng/ml) and EphrinB2 (10 ng/ml) and the higher concentration of GDNF (500 ng/ml) tested were more effectively increased dispersion at later timepoints (Day 15).

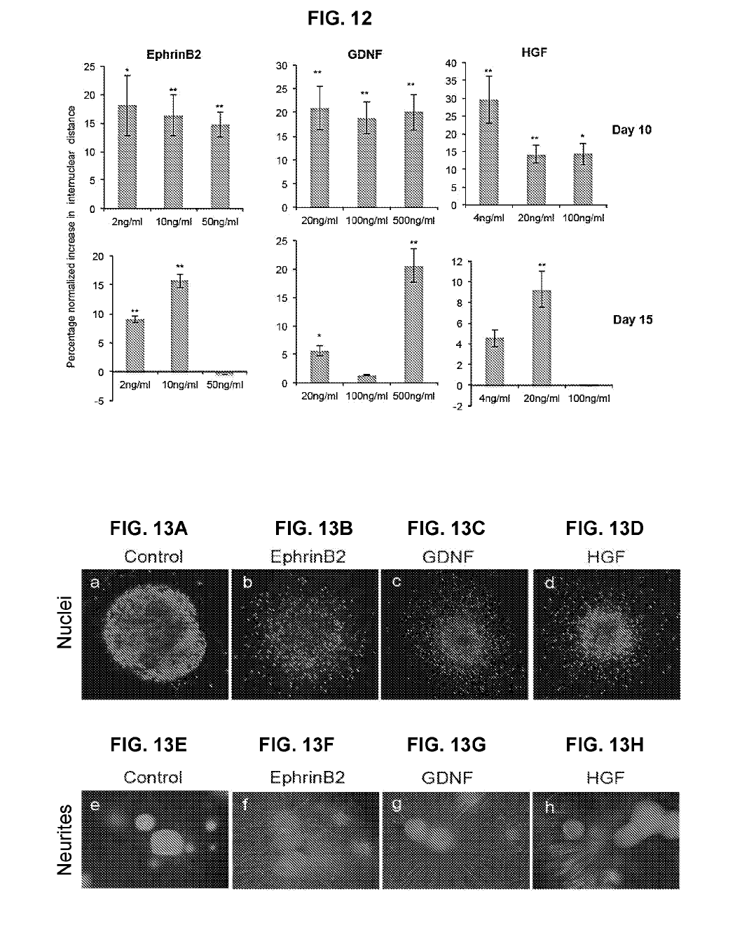

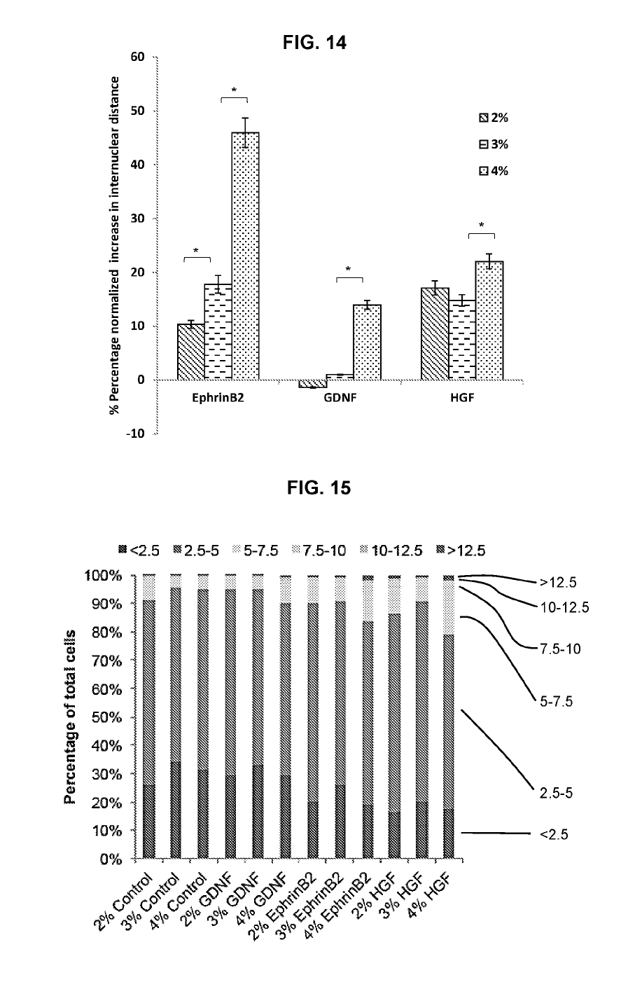

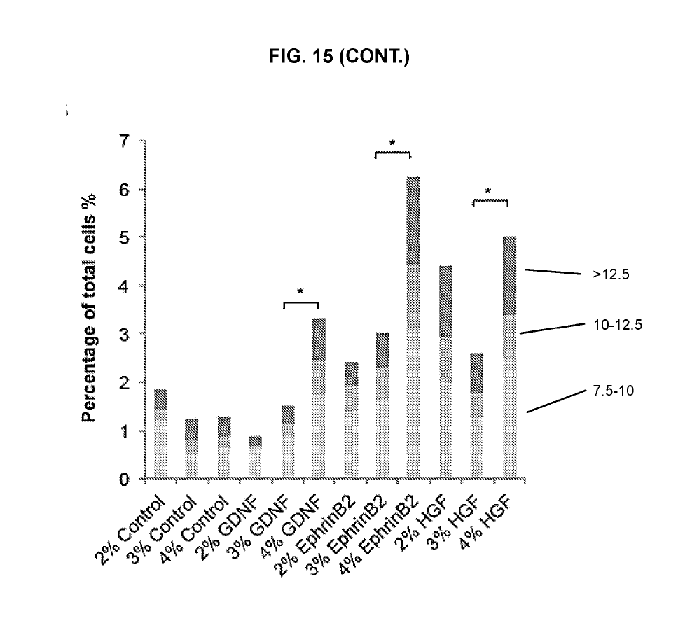

mDA neurons encapsulated in 3D HA hydrogels and treated with HGF, GDNF or EphrinB2 showed extensive neurite outgrowth and dispersion compared to untreated neurons (FIG. 13). These dispersion factors were incorporated into HA gels and different gel stiffnesses were evaluated for an effect on dispersion and other cell characteristics including the promotion of neuronal development and neurite extension (FIG. 14). In depth analysis of dispersing cells showed how different cells within the population were affected differently post-treatment with dispersion factors EphrinB2, GDNF and HGF (FIG. 15). Generally, increased dispersion was seen at higher stiffnesses with the addition of dispersion factors. As an example, increased dispersion and neurite extension of encapsulated cells, e.g., of hESC-derived mDA neurons (20% higher dispersion compared to untreated cells) and hESC derived medium spiny neurons (MSNs) (15% higher dispersion compared to untreated cells), was demonstrated within stiffness-optimized HA gels functionalized with dispersion factors in in vitro cultures.

### FIG. 11:

Dispersion of hPSC derived mDA neurons. Internuclear distances of mDA neurons cultured on 2D laminin coated surfaces, treated with different dispersion factors for 2 days, as percentage increase over untreated cells.

### FIG. 12:

Effect of varying concentrations of factors EphrinB2, GDNF and HGF on dispersion of D25 mDA neurons 10 and 15 days after factor addition. Data is presented as mean±s.e.m. Student's t test was calculated for statistical significance. * p<0.05, ** p<0.005.

### FIG. 13A-13H:

Dispersion of midbrain dopaminergic (mDA) neurons cultured in 3D HA hydrogels and treated with factors. Confocal images demonstrating dispersion of control cells (a), compared to cells treated with EphrinB2 (b), GDNF (c) or HGF (d). Fluorescence images demonstrating neurite extension in control cells (e) compared to cells treated with EphrinB2 (f), GDNF (g) or HGF (h).

### FIG. 14:

Effect of HA gel stiffness on dispersion. Internuclear distances of HA encapsulated mDA neurons treated with dispersion factors as a percentage increase over untreated cells, for hydrogels formulated at 2, 3 and 4 w/v % HA. Data is presented as mean±s.e.m. Student's t test was calculated for statistical significance; * p<0.05.

### FIG. 15:

Effect of increasing HA gel stiffness (at 2, 3 and 4 w/v % HA) on differential dispersion of populations of D25 mDA neurons, with and without addition of factors EphrinB2, GDNF and HGF. Stacked bars represent specific ranges of internuclear distances in μm. The right panel is a subsection of the data presented in the left panel data, focusing on internuclear distances >7.5 μm. Data is presented as mean±s.e.m. Student's t test was calculated for statistical significance; * p<0.05.

Dispersion factor functionalized and stiffness optimized HA hydrogel can improve the post-transplantation integration and dispersion of grafted cells, leading to more efficient cell engraftment and better treatment outcomes. In addition, these modifications may be used to create better 3D in vitro tissue models.

### Example 4

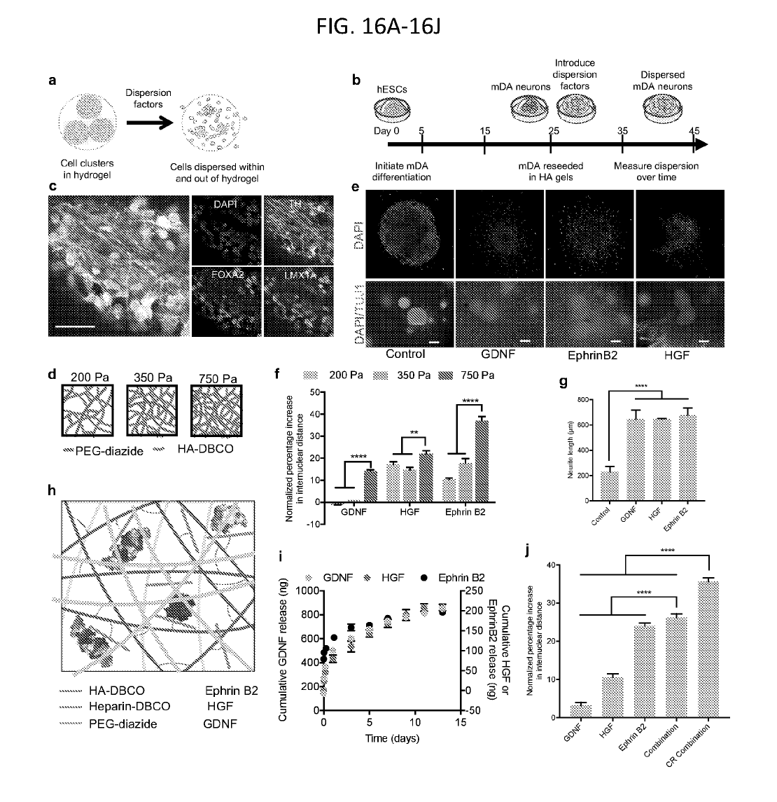

FIG. 16A-16J. hESC-derived mDA neurons are dispersed by biological cues in 3D HA hydrogels. a) Schematic for cell dispersion concept in hydrogels. b) Schematic for generating hESC-derived mDA neurons in 3D PNIPAAm-PEG hydrogels, and transfer to HA hydrogels at D25 for investigating the effect of dispersion factors in 3D. c) Representative immunocytochemistry image showing HA-encapsulated D35 hESC-derived mDA neurons co-expressing TH (cyan), FOXA2 (red) and LMX1A (red), with nuclei labeled in blue. Scale bar is 100 μm. d) Schematic for generating HA gels with higher stiffness by controlling the weight fraction of HA polymers. e) Representative images showing of hESC-derived mDA neurons treated with dispersion factors for 10 days in 750 Pa HA hydrogels. Top panels: dispersed DAPI-labeled nuclei. Bottom panels: Neurite extension, with cells labeled with TUJ1 (red) and DAPI (blue). Scale bar is 200 μm. f) Percentage increase in internuclear distances for hESC-derived mDA neurons treated for ten days with soluble dispersion factors in HA gels of stiffness 200 Pa (orange), 350 Pa (blue) or 750 Pa (purple), relative to internuclear distances for untreated controls. g) Average neurite length in hESC-derived mDA cultures treated with soluble dispersion factors in 750 Pa HA gels. h) Schematic for dispersion factors GDNF, HGF and Ephrin B2 incorporated within HA gels through electrostatic heparin binding. Positively and negatively charged domains are represented in blue and red respectively. i) Release kinetics of dispersion factors GDNF (orange), HGF (purple) and Ephrin B2 (black) from 750 Pa HA gels. j) Percentage increase in internuclear distances for hESC-derived mDA neurons treated for ten days with soluble dispersion factors individually or in combination in 750 Pa HA gels (Combination), or for hESC-derived mDA neurons co-encapsulated with all dispersion factors in 750 Pa HA gels (CR Combination) for ten days, relative to internuclear distances for untreated controls. Data are presented as mean±SEM (n=3). ** indicates p<0.01 and **** indicates p<0.0001 for one way ANOVA with Tukey's test for multiple comparison.

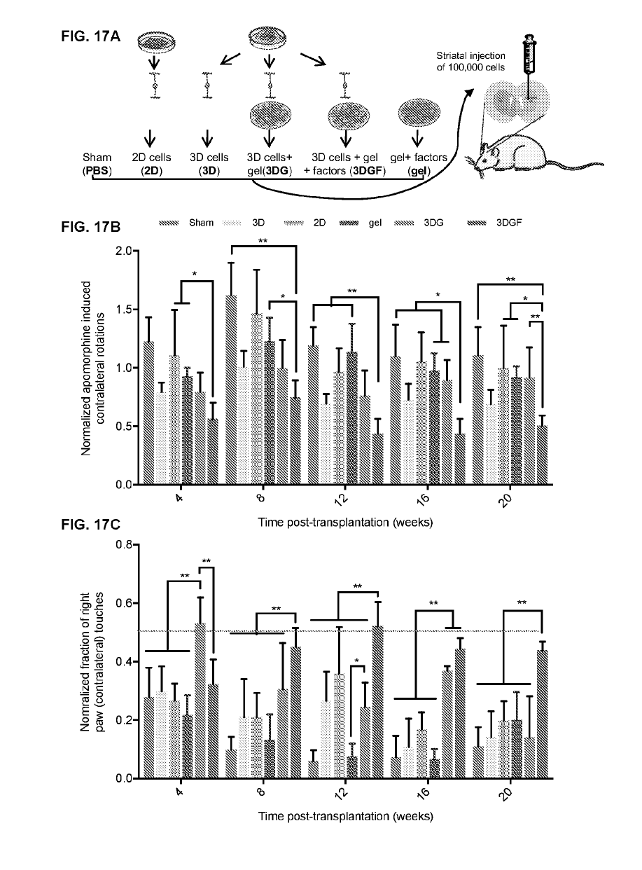

FIG. 17A-17C. Transplantation of hESC-derived mDA neurons co-encapsulated with dispersion factors into 6-OHDA unilesioned PD model rats rapidly alleviates disease symptoms and outperforms alternative treatment groups. a) Schematic representing the six different groups used for in vivo experiments. b) Apomorphine-induced contralateral rotations, normalized to pre-transplantation rotations. c) Fraction of right (contralateral) paw touches in the cylinder test, normalized to fraction of pre-transplantation right paw touches. Dashed red line shows level for full recovery. Groups presented here are: sham (gray), 3D (yellow), 2D (orange), gel (olive), 3D gel (green) and 3D gel+factors (purple). Data are presented as mean±SEM for n=6 for each treatment group, and n=5 for controls. * indicates p<0.05 and ** indicates p<0.01 for one way ANOVA with Tukey's test for multiple comparisons.

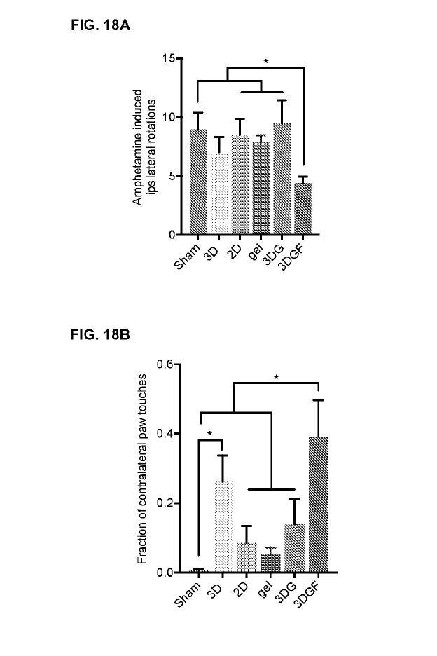

FIG. 18A-18B. 3DGF performs superiorly in additional tests of motor function. a) Net amphetamine-induced contralateral rotations per minute at 20-weeks post-transplantation. b) Fraction of ipsilateral paw touches in the stepping test at 20-weeks post-transplantation. Groups presented here are: sham (gray), 3D (yellow), 2D (orange), gel (olive), 3D gel (green) and 3D gel +factors (purple). Data are presented as mean±SEM for n=6 for each treatment group, and n=5 for controls. * indicates p<0.05 and ** indicates p<0.01 for one way ANOVA with Tukey's test for multiple comparisons.

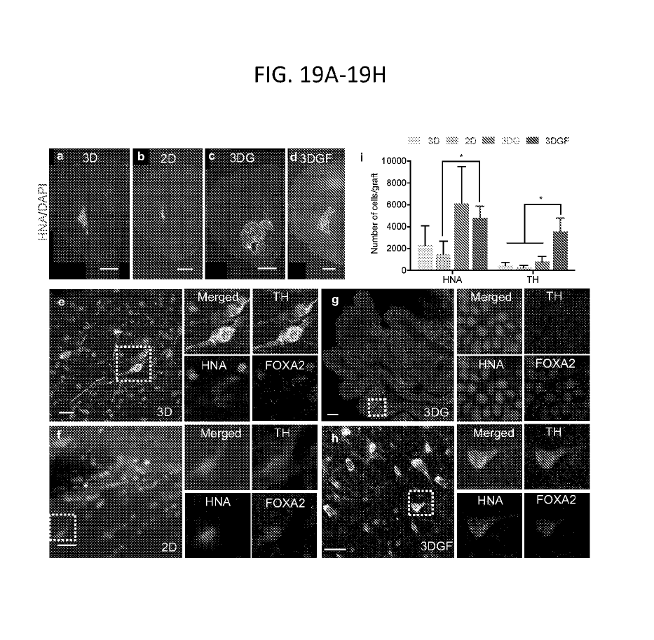

FIG. 19A-19H. HA hydrogels with incorporated dispersion factors demonstrate increased survival and phenotype maintenance of co-transplanted hESC-derived mDA neurons at 20-weeks post-transplantation relative to other treatment groups. a-d) Representative images of HNA⁺ cells (green) in surviving striatal grafts each of the treatment groups. e-h) Representative images of HNA⁺ cells (red) within grafts co-expressing TH (green) and FOXA2 (blue) for each of the treatment groups. Images are representative of n=6 animals/group i) Quantification through immunohistochemistry showing the average number of total and TH⁺ cells surviving for each treatment group: 3D (yellow), 2D (orange), 3D gel (green) and 3D gel+factors (purple). Data are presented as mean±SEM for n=6 animals per group. * indicates p<0.05 for one way ANOVA with Tukey's test for multiple comparisons.

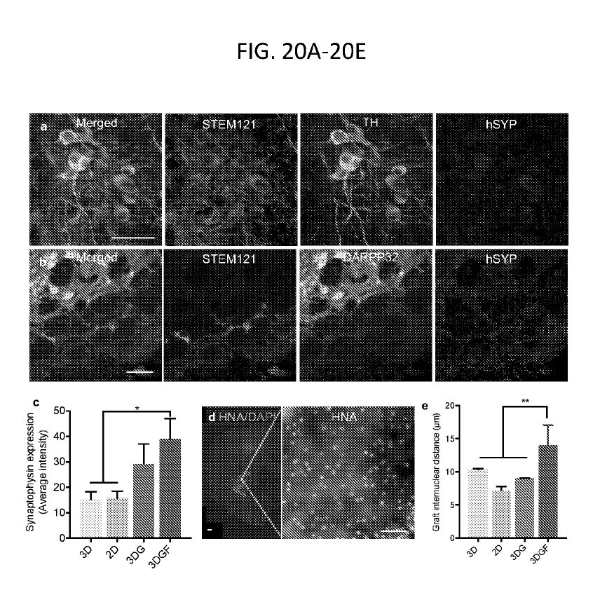

FIG. 20A-20E. hESC-derived mDA neurons transplanted with dispersive hydrogels demonstrate enhanced synaptic integration and dispersion at 20-weeks post-transplantation. a) Representative immunohistochemistry images showing STEM121 labeled human cells co-expressing TH (green) and hSYP (blue) b) Representative images showing STEM121 labeled human cells (red) expressing human synaptophysin (hSYP, shown in blue) in close proximity to host DARPP32⁺ striatal neurons (green) c) Quantification of human synaptophysin levels in neighboring host tissue for each of the treatment groups: 3D (yellow), 2D (orange), 3D gel (green) and 3D gel+factors (purple). d) Representative images of dispersed HNA stained human nuclei (green) for 3DGF. Scale bars 50 μm. e) Average internuclear distance among grafted cells for each of the different treatment groups: 3D (yellow), 2D (orange), 3D gel (green) and 3D gel+factors (purple). Data are presented as mean±SEM for n=6 animals/group. * indicates p<0.05 and ** indicates p<0.01 for one way ANOVA with Tukey's test for multiple comparisons.

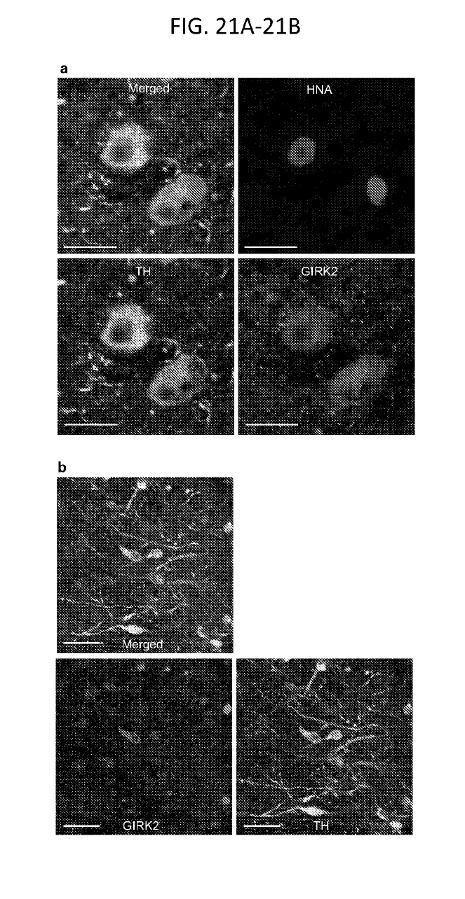

FIG. 21A-21. hESC-derived mDA neurons transplanted with dispersive hydrogels maintain mDA phenotype at 20-weeks post-transplantation a) HNA positive human cells in the graft expressing TH (green) and GIRK 2 (red). b) Surviving TH⁺ neurons in the 3DGF graft express GIRK2. Scale bar is 25 μm.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of delivering a cellularized hydrogel to a subject in need thereof, the method comprising:
contacting a cellular sample with cell-encapsulating hydrogel components to generate a cellularized hydrogel mixture, wherein the cell-encapsulating hydrogel components comprise:
(a) a first backbone polymer comprising hyaluronic acid with an attached azide or cyclooctyne reactive group;
(b) a second backbone polymer comprising heparin with an attached azide or cyclooctyne reactive group;
(c) a cell attachment peptide comprising an azide or cyclooctyne reactive group; and
(d) a linking polymer comprising at least two azide reactive groups or at least two cyclooctyne reactive groups;
incubating the cellularized hydrogel mixture under conditions sufficient to allow cross-linking of the first backbone polymer, the second backbone polymer, the cell attachment peptide and the linking polymer to produce a cellularized hydrogel; and
injecting the cellularized hydrogel into an affected area of the subject, wherein the injecting results in the delivery of a therapeutically effective amount of the cells of the cellular sample into the treatment site of the subject.

2. The method according to claim 1, wherein the cell-encapsulating hydrogel components comprise:
(a) a first backbone polymer comprising hyaluronic acid with an attached cyclooctyne reactive group;
(b) a second backbone polymer comprising heparin with an attached cyclooctyne reactive group;
(c) a cell attachment peptide comprising an azide reactive group; and
(d) a linking polymer comprising at least two azide reactive groups.

3. The method according to claim 1, wherein the cell-encapsulating hydrogel components comprise:
(a) a first backbone polymer comprising hyaluronic acid with an attached azide reactive group;
(b) a second backbone polymer comprising heparin with an attached azide reactive group;
(c) a cell attachment peptide comprising a cyclooctyne reactive group; and
(d) a linking polymer comprising at least two cyclooctyne reactive groups.

4. The method according to claim 1, wherein the linking polymer comprises a polyethylene glycol (PEG) polymer.

5. The method according to claim 4, wherein the linking polymer is a bifunctional PEG azide or a bifunctional PEG cyclooctyne.

6. The method according to any one of the preceding claims, wherein the cell attachment peptide comprises an RGD tripeptide.

7. The method according to claim 6, wherein the cell attachment peptide comprises the amino acid sequence GSGRGDSP (SEQ ID NO:1).

8. The method according to any one of the preceding claims, wherein the cellularized hydrogel mixture comprises a concentration between 0.01 mM and 10 mM of the cell attachment peptide.

9. The method according to claim 8, wherein the concentration of the cell attachment peptide is between 0.1 mM and 1.0 mM.

10. The method according to any one of the preceding claims, wherein the cellularized hydrogel mixture comprises a weight-to-weight percentage of the second backbone polymer to the first backbone polymer between 0.01% and 0.15%.

11. The method according to claim 10, wherein the weight-to-weight percentage of the second backbone polymer to the first backbone polymer between 0.03% and 0.10%.

12. The method according to any one of the preceding claims, wherein the cell-encapsulating hydrogel components further comprise one or more pro-survival factors.

13. The method according to any one of the preceding claims, wherein greater than 5% of the injected cells engraft into the affected area of the subject.

14. The method according to any one of the preceding claims, wherein the cells of the cellular sample comprise neuronal cells.

15. The method according to claim 14, wherein the neuronal cells are midbrain dopaminergic (mDA) neurons.

16. The method according to any one of the preceding claims, wherein the cells of the cellular sample comprise neuronal precursor cells.

17. The method according to claim 16, wherein the neuronal precursor cells are midbrain dopaminergic (mDA) precursor cells.

18. The method according to any one of the preceding claims, wherein the affected area of the subject is the subject's nervous system.

19. The method according to claim 18, wherein the affected area of the subject is the subject's central nervous system.

20. The method according to claim 19, wherein the affected area of the subject is the subject's brain.

21. The method according to any one of the preceding claims, wherein the cells of the cellularized hydrogel maintain a cellular phenotype in the affected area for at least one month following the injection.

22. The method according to claim 21, wherein the cells of the cellularized hydrogel maintain a cellular phenotype in the affected area for at least one four months following the injection.

23. The method according to claim 21, wherein at least 2% of the cells of the cellularized hydrogel maintain the cellular phenotype in the affected area for at least one month following the injection.

24. The method according to claim 22, wherein at least 5% of the cells of the cellularized hydrogel maintain the cellular phenotype in the affected area for at least four months following the injection.

25. The method according to claim 21, wherein the cellular phenotype comprises the expression of one or more cell type markers.

26. The method according to claim 21, wherein the cellular phenotype comprises one or more cellular morphological characteristics or cell population morphological characteristics.

27. The method according to any one of the preceding claims, wherein the method further comprises differentiating pluripotent progenitor cells into neuronal precursor cells or neuronal cells prior to the contacting.

28. The method according to claim 27, wherein the differentiating comprises 2D cell culture.

29. The method according to claim 27, wherein the differentiating comprises 3D cell culture.

30. The method according to any one of the preceding claims, wherein the cell-encapsulating hydrogel components further comprise a dispersion factor.

31. The method according to claim 30, wherein the dispersion factor promotes neurogenesis, neurite extension or a combination thereof.

32. The method according to claim 30, wherein the dispersion factor is selected from the group consisting of hepatocyte growth factor (HGF), glial derived neurotrophic factor (GDNF) and Ephrin-B2 (EFNB2).

33. The method according to claim 32, wherein the dispersion factor is HGF and the cellularized hydrogel mixture comprises between 1 ng/ml and 100 ng/ml HGF.

34. The method according to claim 32, wherein the dispersion factor is GDNF and the cellularized hydrogel mixture comprises between 1 ng/ml and 1 μg/ml GDNF.

35. The method according to claim 32, wherein the dispersion factor is EFNB2 and the cellularized hydrogel mixture comprises between 0.1 ng/ml and 50 ng/ml EFNB2.

36. The method according to claim 30, wherein the dispersion factor is encapsulated in the cellularized hydrogel.

37. The method according to claim 36, wherein the dispersion factor is encapsulated in a controlled release system.

38. The method according to claim 30, wherein the dispersion factor is covalently attached to one or more of the cell-encapsulating hydrogel components (a) through (d).

39. The method according to claim 30, wherein the dispersion factor comprises a cyclooctyne reactive group or an azide reactive group and the incubating comprises conditions sufficient to allow attachment of the dispersion factor to one or more of the cell-encapsulating hydrogel components (a) through (d).

40. The method according to any one of the preceding claims, wherein the cellularized hydrogel has a gel stiffness that promotes dispersion of the cells of the cellular sample into the treatment site of the subject.

41. The method according to claim 40, wherein the cellularized hydrogel is formulated with a weight-to-volume percentage of the first backbone polymer between 1% and 10%.

42. The method according to claim 41, wherein the cellularized hydrogel is formulated with a weight-to-volume percentage of the first backbone polymer between 2% and 5%.

43. The method according to any one of claims 30 to 42, wherein the cell-encapsulating hydrogel components comprise two or more dispersion factors.

44. The method according to claim 43, wherein two or more dispersion factors are selected from the group consisting of HGF, GDNF and EFNB2.

45. A cellularized hydrogel, the cellular hydrogel comprising:
(a) a first backbone polymer comprising hyaluronic acid;
(b) a second backbone polymer comprising heparin;
(c) a linking polymer;
(d) a dispersion factor; and
(e) a plurality of cells responsive to the dispersion factor, wherein the first backbone polymer and the second backbone polymer are each linked to the linking polymer.

46. The cellularized hydrogel of claim 45, wherein the first backbone polymer and the second backbone polymer are each linked to the linking polymer by a triazole moiety.

47. The cellularized hydrogel of any one of claims 45 to 46, wherein the dispersion factor is encapsulated in the cellularized hydrogel.

48. The cellularized hydrogel of claim 47, wherein the dispersion factor is encapsulated in a controlled release system.

49. The cellularized hydrogel of any one of claims 45 to 46, wherein the dispersion factor is covalently attached to one or more of (a) through (c).

50. The cellularized hydrogel of claim 49, wherein the dispersion factor is covalently attached to one or more of (a) through (c) by a triazole moiety.

51. The cellularized hydrogel of any one of claims 45 to 50, wherein the dispersion factor is selected from the group consisting of hepatocyte growth factor (HGF), glial derived neurotrophic factor (GDNF) and Ephrin-B2 (EFNB2).

52. The cellularized hydrogel of any one of claims 45 to 51, wherein the cellularized hydrogel comprises two or more dispersion factors.

53. The cellularized hydrogel of claim 52, wherein the two or more dispersion factors are selected from the group consisting of HGF, GDNF and EFNB2.

54. The cellularized hydrogel of any one of claims 45 to 53, wherein the cellularized hydrogel comprises between 1 ng/ml and 100 ng/ml HGF.

55. The cellularized hydrogel of any one of claims 45 to 53, wherein the cellularized hydrogel comprises between 1 ng/ml and 1 μg/ml GDNF.

56. The cellularized hydrogel of any one of claims 45 to 53, wherein the cellularized hydrogel between 0.1 ng/ml and 50 ng/ml EFNB2.

57. The cellularized hydrogel of any one of claims 45 to 56, wherein the dispersion factor promotes neurogenesis, neurite extension or a combination thereof.

58. The cellularized hydrogel of any one of claims 45 to 57, wherein the linking polymer comprises a polyethylene glycol (PEG) polymer.

59. The cellularized hydrogel of claim 58, wherein the linking polymer is a bifunctional PEG azide or a bifunctional PEG cyclooctyne.

60. The cellularized hydrogel of any one of claims 45 to 59, wherein the cellularized hydrogel further comprises a cell attachment peptide covalently attached to one or more of (a) through (c).

61. The cellularized hydrogel of claim 60, wherein the cell attachment peptide is covalently attached to one or more of (a) through (c) by a triazole moiety.

62. The cellularized hydrogel of any one of claims 60 to 61, wherein the cell attachment peptide comprises an RGD tripeptide.

63. The cellularized hydrogel of claim 62, wherein the cell attachment peptide comprises the amino acid sequence GSGRGDSP (SEQ ID NO:1).

64. The cellularized hydrogel of any one of claims 60 to 63, wherein the cellularized hydrogel comprises a concentration between 0.01 mM and 10 mM of the cell attachment peptide.

65. The cellularized hydrogel of claim 64, wherein the concentration of the cell attachment peptide is between 0.1 mM and 1.0 mM.

66. The cellularized hydrogel of any one of claims 45 to 65, wherein the cellularized hydrogel mixture comprises a weight-to-weight percentage of the second backbone polymer to the first backbone polymer between 0.01% and 0.15%.

67. The cellularized hydrogel of claim 66, wherein the weight-to-weight percentage of the second backbone polymer to the first backbone polymer between 0.03% and 0.10%.

68. The cellularized hydrogel of any one of claims 45 to 67, wherein the cellularized hydrogel further comprises one or more pro-survival factors.

69. The cellularized hydrogel of any one of claims 45 to 68, wherein the plurality of cells comprise neuronal cells.

70. The cellularized hydrogel of claim 69, wherein the neuronal cells comprise midbrain dopaminergic (mDA) neurons.

71. The cellularized hydrogel of any one of claims 45 to 68, wherein the plurality of cells comprise neuronal precursor cells.

72. The cellularized hydrogel of claim 71, wherein the neuronal precursor cells comprise midbrain dopaminergic (mDA) precursor cells.

73. The cellularized hydrogel of any one of claims 45 to 72, wherein the cellularized hydrogel has a gel stiffness that promotes dispersion of the plurality of cells.

74. The cellularized hydrogel of claim 73, wherein the cellularized hydrogel is formulated with a weight-to-volume percentage of the first backbone polymer between 1% and 10%.

75. The cellularized hydrogel of claim 74, wherein the cellularized hydrogel is formulated with a weight-to-volume percentage of the first backbone polymer between 2% and 5%.

76. A kit for making a cellularized hydrogel, the kit comprising:
(a) a first backbone polymer comprising hyaluronic acid;
(b) a second backbone polymer comprising heparin;
(c) a linking polymer; and
(d) a dispersion factor, wherein at least the linking polymer is in a separate container.

77. The kit according to claim 76, further comprising a cell attachment peptide.

78. The kit according to any one of claims 76 to 77, wherein the first backbone polymer and the second backbone polymer each comprise an attached cyclooctyne reactive group and the linking polymer comprises at least two azide reactive groups.

79. The kit according to any one of claims 76 to 77, wherein the first backbone polymer and the second backbone polymer each comprise an attached azide reactive group and the linking polymer comprises at least two cyclooctyne reactive groups.

80. The kit according to any one of claims 76 to 79, wherein the dispersion factor comprises an attached cyclooctyne reactive group or an attached azide reactive group.

81. The kit according to claim 77, wherein cell attachment peptide comprises an attached cyclooctyne reactive group or an attached azide reactive group.

82. The kit according to any one of claims 76 to 81, wherein the kit is configured for the preparation of a therapeutic cellularized hydrogel.

83. The kit according to any one of claims 76 to 81, wherein the kit is configured for the preparation of an in vitro tissue model cellularized hydrogel.


SEQUENCE LISTING (38 sequences)

SEQ ID NO: 1 - protein, 8 aa, Artificial Sequence
  Synthetic sequence
GSGRGDSP

SEQ ID NO: 2 - protein, 8 aa, Artificial Sequence
  Synthetic sequence
GSGRGDSP

SEQ ID NO: 3 - protein, 5 aa, Artificial Sequence
  Synthetic sequence
GFGER

SEQ ID NO: 4 - protein, 5 aa, Artificial Sequence
  Synthetic sequence
YIGSR

SEQ ID NO: 5 - protein, 5 aa, Artificial Sequence
  Synthetic sequence
IKVAV

SEQ ID NO: 6 - protein, 5 aa, Artificial Sequence
  Synthetic sequence
PDSGR

SEQ ID NO: 7 - protein, 15 aa, Artificial Sequence
  Synthetic sequence
GGGNGEPRGDTYRAY

SEQ ID NO: 8 - protein, 5 aa, Artificial Sequence
  Synthetic sequence
PHSRN

SEQ ID NO: 9 - protein, 4 aa, Artificial Sequence
  Synthetic sequence
REDV

SEQ ID NO: 10 - protein, 6 aa, Artificial Sequence
  Synthetic sequence
GRGDSP

SEQ ID NO: 11 - DNA, 20 bp, Artificial Sequence
  Synthetic sequence
caccatctgtcgcttcgagg

SEQ ID NO: 12 - DNA, 22 bp, Artificial Sequence
  Synthetic sequence
agggtctccgatttgcatatct

SEQ ID NO: 13 - DNA, 21 bp, Artificial Sequence
  Synthetic sequence
aaggtcccggtcaagaaacag

SEQ ID NO: 14 - DNA, 20 bp, Artificial Sequence
  Synthetic sequence
cttctgcgtcacaccattgc

SEQ ID NO: 15 - DNA, 22 bp, Artificial Sequence
  Synthetic sequence
aacgataacataccaagcgtgt

SEQ ID NO: 16 - DNA, 19 bp, Artificial Sequence
  Synthetic sequence
ggtctgcccgttcaacatc

SEQ ID NO: 17 - DNA, 21 bp, Artificial Sequence
  Synthetic sequence
ggagcagctactatgcagagc

SEQ ID NO: 18 - DNA, 20 bp, Artificial Sequence
  Synthetic sequence
cgtgttcatgccgttcatcc

SEQ ID NO: 19 - DNA, 21 bp, Artificial Sequence
  Synthetic sequence
catgcagaggtcctatcccat

SEQ ID NO: 20 - DNA, 21 bp, Artificial Sequence
  Synthetic sequence
aagctggggactgattgagat

SEQ ID NO: 21 - DNA, 20 bp, Artificial Sequence
  Synthetic sequence
cctacgaggaggtgtacccc

SEQ ID NO: 22 - DNA, 19 bp, Artificial Sequence
  Synthetic sequence
cccacgttgaccgagttga

SEQ ID NO: 23 - DNA, 21 bp, Artificial Sequence
  Synthetic sequence
tttctcctgtccgtcattggc

SEQ ID NO: 24 - DNA, 21 bp, Artificial Sequence
  Synthetic sequence
agcccacacctttcagtatgg

SEQ ID NO: 25 - DNA, 19 bp, Artificial Sequence
  Synthetic sequence
gggctgtgtaagcagaacg

SEQ ID NO: 26 - DNA, 19 bp, Artificial Sequence
  Synthetic sequence
aaggcccgaatctcaggct

SEQ ID NO: 27 - DNA, 21 bp, Artificial Sequence
  Synthetic sequence
accactcttcgggagaataca

SEQ ID NO: 28 - DNA, 21 bp, Artificial Sequence
  Synthetic sequence
ggcatttggtacaagcaaggt

SEQ ID NO: 29 - DNA, 20 bp, Artificial Sequence
  Synthetic sequence
cacatcagccgagatcggac

SEQ ID NO: 30 - DNA, 21 bp, Artificial Sequence
  Synthetic sequence
ggtagcgataggtctccctca

SEQ ID NO: 31 - DNA, 19 bp, Artificial Sequence
  Synthetic sequence
ggccaagggtcactacacg

SEQ ID NO: 32 - DNA, 21 bp, Artificial Sequence
  Synthetic sequence
gcagtcgcagttttcacactc

SEQ ID NO: 33 - DNA, 21 bp, Artificial Sequence
  Synthetic sequence
acgtccgagaaccatcttgac

SEQ ID NO: 34 - DNA, 19 bp, Artificial Sequence
  Synthetic sequence
caccaccgtttgtctgagc

SEQ ID NO: 35 - DNA, 19 bp, Artificial Sequence
  Synthetic sequence
gagcgcagggcaccaaata

SEQ ID NO: 36 - DNA, 21 bp, Artificial Sequence
  Synthetic sequence
aataacgtgtgcagtacaccc

SEQ ID NO: 37 - DNA, 20 bp, Artificial Sequence
  Synthetic sequence
ggagcgagatccctccaaat

SEQ ID NO: 38 - DNA, 23 bp, Artificial Sequence
  Synthetic sequence
ggctgttgtcatacttctcatgg